SEQUENCE LISTING (231 sequences)

SEQ ID NO: 1 - protein, 15 aa, Homo sapiens
  REGION 1..15: note = MISC_Feature - CDR3 alpha
CAVHSTAQAGTALIF

SEQ ID NO: 2 - protein, 16 aa, Homo sapiens
  REGION 1..16: note = MISC_feature - CDR3 beta
CASSTHRGQTNYGYTF

SEQ ID NO: 3 - protein, 7 aa, Homo sapiens
  REGION 1..7: note = MISC_feature - CDR2 alpha
LYSAGEE

SEQ ID NO: 4 - protein, 6 aa, Homo sapiens
  REGION 1..6: note = MISC_feature - CDR2 beta
YYNGEE

SEQ ID NO: 5 - protein, 6 aa, Homo sapiens
  REGION 1..6: note = MISC_feature - CDR1 alpha
VSGLRG

SEQ ID NO: 6 - protein, 5 aa, Homo sapiens
  REGION 1..5: note = MISC_feature - CDR1 beta
SGDLS

SEQ ID NO: 7 - protein, 274 aa, Homo sapiens
  REGION 1..274: note = MISC_feature - TCR alpha wt full length
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSTAQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 8 - protein, 311 aa, Homo sapiens
  REGION 1..311: note = MISC_feature - TCR beta wt full length
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTHRGQTNYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 9 - protein, 274 aa, synthetic construct; identical to SEQ ID NO: 7
  REGION 1..274: note = TCR alpha full length humC

SEQ ID NO: 10 - protein, 311 aa, synthetic construct; identical to SEQ ID NO: 8
  REGION 1..311: note = TCR beta humC full length

SEQ ID NO: 11 - protein, 274 aa, synthetic construct
  REGION 1..274: note = TCR alpha mmcys full length
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSTAQAGTALIFGKGTTLSVSSNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 12 - protein, 311 aa, synthetic construct
  REGION 1..311: note = TCR beta mmcys full length
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTHRGQTNYGYTFGSGTRLTVVEDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 13 - protein, 270 aa, synthetic construct
  REGION 1..270: note = TCR alpha murC full length
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSTAQAGTALIFGKGTTLSVSSNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 14 - protein, 307 aa, synthetic construct
  REGION 1..307: note = TCR beta murC full length
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTHRGQTNYGYTFGSGTRLTVVEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 15 - protein, 134 aa, Homo sapiens
  REGION 1..134: note = MISC_feature - TCR alpha variable
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVHSTAQAGTALIFGKGTTLSVSSN

SEQ ID NO: 16 - protein, 134 aa, synthetic construct
  REGION 1..134: note = TCR beta variable
  REGION 1..134: note = MISC_feature - TCR beta variable
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSTHRGQTNYGYTFGSGTRLTVV

SEQ ID NO: 17 - protein, 140 aa, Homo sapiens
  REGION 1..140: note = MISC_FEATURE - TCR alpha wt constant
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 177 aa, Homo sapiens
  REGION 1..177: note = MISC_FEATURE - TCR beta wt constant
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 19 - protein, 140 aa, synthetic construct
  REGION 1..140: note = TCR alpha minimal murinized constant
  REGION 1..140: note = MISC_feature - TCR alpha mmcys constant
IQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 20 - protein, 177 aa, synthetic construct
  REGION 1..177: note = TCR beta minimally murinized C
  REGION 1..177: note = MISC_feature - TCR beta mmcys constant
EDLNKVFPPEVAVFEPSKAEIAHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 21 - protein, 136 aa, Mus musculus
  REGION 1..136: note = MISC_FEATURE - TCR alpha murC constant
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 22 - protein, 173 aa, Mus musculus
  REGION 1..173: note = MISC_feature - TCR beta murC constant
EDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 23 - DNA, 825 bp, Homo sapiens
  misc_feature 1..825: note = MISC_feature - TCR alpha wt
  source 1..825: mol_type = genomic DNA
atggagaaaatgttggagtgtgcattcatagtcttgtggcttcagcttggctggttgagtggagaagaccaggtgacgcagagtcccgaggccctgagactccaggagggagagagtagcagtcttaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcactccacagcccaggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 24 - DNA, 936 bp, Homo sapiens
  misc_feature 1..936: note = MISC_feature - TCR beta wt
  source 1..936: mol_type = genomic DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcacacacagggggcagactaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 25 - DNA, 825 bp, synthetic construct
  misc_feature 1..825: note = TCR alpha humC codon-optimized
  source 1..825: mol_type = other DNA
atggaaaagatgctggaatgcgccttcatcgtgctgtggctgcagctgggatggctgagcggagaggaccaagtgacccagtctcccgaggccctgagactgcaggaaggcgagagcagcagcctgaactgcagctacaccgtgtccggcctgagaggcctgttctggtacagacaggaccccggcaagggccccgagttcctgttcacactgtactctgccggcgaggaaaaagagaaagagcggctgaaggccaccctgaccaagaaagagagcttcctgcacatcaccgcccccaagcctgaggacagcgccacatatctgtgcgccgtgcactctacagcccaggccggaaccgccctgatctttggcaagggcaccaccctgagcgtgtccagcaacatccagaatccggaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 26 - DNA, 936 bp, synthetic construct
  misc_feature 1..936: note = TCR beta humC codon-optimized
  source 1..936: mol_type = other DNA
atgggctttagactgctgtgctgcgtggccttctgcctgctgggagctggccctgtggatagcggcgtgacccagacacccaagcacctgatcacagccaccggccagcgcgtgaccctgagatgttctcctagaagcggcgacctgagcgtgtactggtatcagcagagcctggaccagggcctgcagttcctgatccagtactacaacggcgaggaacgggccaagggcaacatcctggaacggttcagcgcccagcagttccccgatctgcacagcgagctgaacctgagcagcctggaactgggcgacagcgccctgtacttctgtgccagcagcacccacagaggccagaccaactacggctacaccttcggcagcggcaccagactgaccgtggtggaggacctgaacaaagtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 27 - DNA, 825 bp, synthetic construct
  misc_feature 1..825: note = TCR alpha mmcys codon-optimized
  source 1..825: mol_type = other DNA
atggaaaagatgctggaatgcgccttcatcgtgctgtggctgcagctgggatggctgagcggagaggaccaagtgacccagtctcccgaggccctgagactgcaggaaggcgagagcagcagcctgaattgcagctacaccgtgtccggcctgcggggcctgttctggtatagacaggaccctggcaagggccccgagttcctgtttaccctgtactctgccggcgaagagaaagaaaaagagcggctgaaggccacactgaccaagaaagagagcttcctgcacatcaccgcccccaagcctgaggacagcgccacatatctgtgcgccgtgcactctacagcccaggccggaaccgccctgatcttcggcaagggcacaaccctgtccgtgtccagcaacatccagaaccccgaccccgccgtgtaccagctgagagacagcaagagcagcgacaagagcgtgtgcctgttcaccgacttcgacagccagacaaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttccccagctccgacgtgccctgcgacgtgaagctggtggaaaagagcttcgagacagacaccaacctgaacttccagaatctgagcgtgatcggcttcagaatcctgctgctgaaggtggccggcttcaatctgctgatgaccctgcggctgtggtccagctga

SEQ ID NO: 28 - DNA, 935 bp, synthetic construct
  misc_feature 1..935: note = TCR beta mmcys codon-optimized
  source 1..935: mol_type = other DNA
atgggcttccggctgctgtgttgcgtggccttctgtctgctgggagccggccctgtggatagcggcgtgacacagacacccaagcacctgatcaccgccaccggccagcgcgtgacactgagatgtagccctagaagcggcgacctgagcgtgtactggtatcagcagagcctggaccagggcctgcagttcctgatccagtactacaacggcgaggaacgggccaagggcaacatcctggaacggttcagcgcccagcagttccccgatctgcacagcgagctgaacctgagcagcctggaactgggcgacagcgccctgtacttctgtgccagcagcacccacagaggccagaccaactacggctacaccttcggcagcggcaccagactgaccgtggtggaagatctgaacaaggtgttccccccagaggtggccgtgttcgagcctagcaaggccgagatcgcccacacccagaaagccaccctcgtgtgtctggccaccggctttttccccgaccacgtggaactgtcttggtgggtcaacggcaaagaggtgcactccggcgtgtgcaccgatccccagcctctgaaagaacagcccgccctgaacgacagccggtactgcctgtccagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgtggcatcaccagcgccagctaccatcagggcgtgctgagcgccaccatcctgtacgagatcctgctgggcaaggccaccctgtacgccgtgctggtgtctgccctggtgctgatggccatggtcaagcggaaggactttga

SEQ ID NO: 29 - DNA, 813 bp, synthetic construct
  misc_feature 1..813: note = TCR alpha murC codon-optimized
  source 1..813: mol_type = other DNA
atggaaaagatgctggaatgcgccttcatcgtgctgtggctgcagctgggatggctgagcggagaggaccaagtgacccagtctcccgaggccctgagactgcaggaaggcgagagcagcagcctgaactgcagctacaccgtgtccggcctgagaggcctgttctggtacagacaggaccccggcaagggccccgagttcctgttcacactgtactctgccggcgaggaaaaagagaaagagcggctgaaggccaccctgaccaagaaagagagcttcctgcacatcaccgcccccaagcctgaggacagcgccacatatctgtgcgccgtgcactctacagcccaggccggaaccgccctgatctttggcaagggcaccaccctgagcgtgtccagcaacatccagaaccccgagcccgccgtgtaccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctga

SEQ ID NO: 30 - DNA, 924 bp, synthetic construct
  misc_feature 1..924: note = TCR beta murC codon-optimized
  source 1..924: mol_type = other DNA
atgggctttagactgctgtgctgcgtggccttctgcctgctgggagctggccctgtggatagcggcgtgacccagacacccaagcacctgatcacagccaccggccagcgcgtgaccctgagatgttctcctagaagcggcgacctgagcgtgtactggtatcagcagagcctggaccagggcctgcagttcctgatccagtactacaacggcgaggaacgggccaagggcaacatcctggaacggttcagcgcccagcagttccccgatctgcacagcgagctgaacctgagcagcctggaactgggcgacagcgccctgtacttctgtgccagcagcacccacagaggccagaccaactacggctacaccttcggcagcggcaccagactgaccgtggtggaagatctgcggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctga

SEQ ID NO: 31 - protein, 9 aa, synthetic construct
  REGION 1..9: note = MINIMAL RECOGNITION SEQUENCE
  VARIANT 1: note = X can be selected from any amino acid
  VARIANT 3: note = X can be selected from any amino acid
  VARIANT 7: note = X can be selected from any amino acid
XLXGLDXLL

SEQ ID NO: 32 - protein, 9 aa, Homo sapiens
VLDGLDVLL

SEQ ID NO: 33 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 34 - protein, 6 aa, Homo sapiens
TSGFYG

SEQ ID NO: 35 - protein, 6 aa, Homo sapiens
TSGFNG

SEQ ID NO: 36 - protein, 6 aa, Homo sapiens
VSNAYN

SEQ ID NO: 37 - protein, 6 aa, Homo sapiens
VSGNPY

SEQ ID NO: 38 - protein, 7 aa, Homo sapiens
NIATNDY

SEQ ID NO: 39 - protein, 6 aa, Homo sapiens
DSSSTY

SEQ ID NO: 40 - protein, 6 aa, Homo sapiens
NYSPAY

SEQ ID NO: 41 - protein, 6 aa, Homo sapiens
VSRFNN

SEQ ID NO: 42 - protein, 6 aa, Homo sapiens
YGGTVN

SEQ ID NO: 43 - protein, 6 aa, Homo sapiens
SSYSPS

SEQ ID NO: 44 - protein, 6 aa, Homo sapiens
YGATPY

SEQ ID NO: 45 - protein, 6 aa, Homo sapiens
SSVPPY

SEQ ID NO: 46 - protein, 6 aa, Homo sapiens
SSVSVY

SEQ ID NO: 47 - protein, 6 aa, Homo sapiens
TTQYPS

SEQ ID NO: 48 - protein, 6 aa, Homo sapiens
ATGYPS

SEQ ID NO: 49 - protein, 6 aa, Homo sapiens
VSPFSN

SEQ ID NO: 50 - protein, 6 aa, Homo sapiens
ERTLFN

SEQ ID NO: 51 - protein, 6 aa, Homo sapiens
NSASQS

SEQ ID NO: 52 - protein, 6 aa, Homo sapiens
DRGSQS

SEQ ID NO: 53 - protein, 6 aa, Homo sapiens
NSAFQY

SEQ ID NO: 54 - protein, 6 aa, Homo sapiens
DSASNY

SEQ ID NO: 55 - protein, 6 aa, Homo sapiens
NSASDY

SEQ ID NO: 56 - protein, 7 aa, Homo sapiens
TSDPSYG

SEQ ID NO: 57 - protein, 6 aa, Homo sapiens
YSGSPE

SEQ ID NO: 58 - protein, 5 aa, Homo sapiens
TSINN

SEQ ID NO: 59 - protein, 6 aa, Homo sapiens
SSYSTF

SEQ ID NO: 60 - protein, 7 aa, Homo sapiens
TRDTTYY

SEQ ID NO: 61 - protein, 6 aa, Homo sapiens
VSGLRG

SEQ ID NO: 62 - protein, 6 aa, Homo sapiens
DSAIYN

SEQ ID NO: 63 - protein, 5 aa, Homo sapiens
DSVNN

SEQ ID NO: 64 - protein, 6 aa, Homo sapiens
NTAFDY

SEQ ID NO: 65 - protein, 6 aa, Homo sapiens
SSNFYA

SEQ ID NO: 66 - protein, 5 aa, Homo sapiens
TTLSN

SEQ ID NO: 67 - protein, 7 aa, Homo sapiens
TISGNEY

SEQ ID NO: 68 - protein, 7 aa, Homo sapiens
TISGTDY

SEQ ID NO: 69 - protein, 5 aa, Homo sapiens
SVFSS

SEQ ID NO: 70 - protein, 6 aa, Homo sapiens
NSMFDY

SEQ ID NO: 71 - protein, 5 aa, Homo sapiens
KALYS

SEQ ID NO: 72 - protein, 5 aa, Homo sapiens
KTLYG

SEQ ID NO: 73 - protein, 5 aa, Homo sapiens
SIFNT

SEQ ID NO: 74 - protein, 6 aa, Homo sapiens
VTNFRS

SEQ ID NO: 75 - protein, 7 aa, Homo sapiens
TSENNYY

SEQ ID NO: 76 - protein, 7 aa, Homo sapiens
TSESDYY

SEQ ID NO: 77 - protein, 5 aa, Homo sapiens
TTSDR

SEQ ID NO: 78 - protein, 6 aa, Homo sapiens
STGYPT

SEQ ID NO: 79 - protein, 5 aa, Homo sapiens
VGISA

SEQ ID NO: 80 - protein, 5 aa, Homo sapiens
SNHLY

SEQ ID NO: 81 - protein, 5 aa, Homo sapiens
LGHDT

SEQ ID NO: 82 - protein, 5 aa, Homo sapiens
MGHRA

SEQ ID NO: 83 - protein, 5 aa, Homo sapiens
LGHNA

SEQ ID NO: 84 - protein, 5 aa, Homo sapiens
LGHNA

SEQ ID NO: 85 - protein, 5 aa, Homo sapiens
SGHRS

SEQ ID NO: 86 - protein, 5 aa, Homo sapiens
SGHNT

SEQ ID NO: 87 - protein, 5 aa, Homo sapiens
SGHKS

SEQ ID NO: 88 - protein, 5 aa, Homo sapiens
SGHDT

SEQ ID NO: 89 - protein, 5 aa, Homo sapiens
SGHTS

SEQ ID NO: 90 - protein, 5 aa, Homo sapiens
MNHNS

SEQ ID NO: 91 - protein, 5 aa, Homo sapiens
MNHEY

SEQ ID NO: 92 - protein, 5 aa, Homo sapiens
MNHEY

SEQ ID NO: 93 - protein, 5 aa, Homo sapiens
MRHNA

SEQ ID NO: 94 - protein, 5 aa, Homo sapiens
MNHEY

SEQ ID NO: 95 - protein, 5 aa, Homo sapiens
MNHNY

SEQ ID NO: 96 - protein, 5 aa, Homo sapiens
MNHGY

SEQ ID NO: 97 - protein, 5 aa, Homo sapiens
MNHGY

SEQ ID NO: 98 - protein, 5 aa, Homo sapiens
SGHTA

SEQ ID NO: 99 - protein, 5 aa, Homo sapiens
SGHTA

SEQ ID NO: 100 - protein, 5 aa, Homo sapiens
SGHVT

SEQ ID NO: 101 - protein, 5 aa, Homo sapiens
SGHVS

SEQ ID NO: 102 - protein, 5 aa, Homo sapiens
SSHAT

SEQ ID NO: 103 - protein, 5 aa, Homo sapiens
SGHVS

SEQ ID NO: 104 - protein, 5 aa, Homo sapiens
SEHNR

SEQ ID NO: 105 - protein, 5 aa, Homo sapiens
SGDLS

SEQ ID NO: 106 - protein, 5 aa, Homo sapiens
WNHNN

SEQ ID NO: 107 - protein, 5 aa, Homo sapiens
WSHSY

SEQ ID NO: 108 - protein, 5 aa, Homo sapiens
ENHRY

SEQ ID NO: 109 - protein, 5 aa, Homo sapiens
SGHAT

SEQ ID NO: 110 - protein, 5 aa, Homo sapiens
SGHAT

SEQ ID NO: 111 - protein, 5 aa, Homo sapiens
SGHNT

SEQ ID NO: 112 - protein, 5 aa, Homo sapiens
SGHND

SEQ ID NO: 113 - protein, 5 aa, Homo sapiens
FGHNF

SEQ ID NO: 114 - protein, 5 aa, Homo sapiens
SGHNS

SEQ ID NO: 115 - protein, 5 aa, Homo sapiens
SGHDY

SEQ ID NO: 116 - protein, 5 aa, Homo sapiens
LGHNT

SEQ ID NO: 117 - protein, 5 aa, Homo sapiens
PRHDT

SEQ ID NO: 118 - protein, 5 aa, Homo sapiens
SGHDN

SEQ ID NO: 119 - protein, 5 aa, Homo sapiens
LNHNV

SEQ ID NO: 120 - protein, 5 aa, Homo sapiens
KGHSY

SEQ ID NO: 121 - protein, 5 aa, Homo sapiens
KGHSH

SEQ ID NO: 122 - protein, 5 aa, Homo sapiens
LNHDA

SEQ ID NO: 123 - protein, 6 aa, Homo sapiens
DFQATT

SEQ ID NO: 124 - protein, 5 aa, Homo sapiens
KGHDR

SEQ ID NO: 125 - protein, 5 aa, Homo sapiens
MGHDK

SEQ ID NO: 126 - protein, 5 aa, Homo sapiens
MNHEY

SEQ ID NO: 127 - protein, 5 aa, Homo sapiens
MDHEN

SEQ ID NO: 128 - protein, 5 aa, Homo sapiens
SQVTM

SEQ ID NO: 129 - protein, 6 aa, Homo sapiens
GTSNPN

SEQ ID NO: 130 - protein, 6 aa, Homo sapiens
NALDGL

SEQ ID NO: 131 - protein, 6 aa, Homo sapiens
NVLDGL

SEQ ID NO: 132 - protein, 4 aa, Homo sapiens
GSKP

SEQ ID NO: 133 - protein, 8 aa, Homo sapiens
YITGDNLV

SEQ ID NO: 134 - protein, 5 aa, Homo sapiens
GYKTK

SEQ ID NO: 135 - protein, 7 aa, Homo sapiens
IFSNMDM

SEQ ID NO: 136 - protein, 7 aa, Homo sapiens
IRENEKE

SEQ ID NO: 137 - protein, 7 aa, Homo sapiens
MYSAGYE

SEQ ID NO: 138 - protein, 8 aa, Homo sapiens
YFSGDPLV

SEQ ID NO: 139 - protein, 8 aa, Homo sapiens
YTSAATLV

SEQ ID NO: 140 - protein, 8 aa, Homo sapiens
YFSGDTLV

SEQ ID NO: 141 - protein, 8 aa, Homo sapiens
YTSAATLV

SEQ ID NO: 142 - protein, 8 aa, Homo sapiens
YLSGSTLV

SEQ ID NO: 143 - protein, 7 aa, Homo sapiens
AMKANDK

SEQ ID NO: 144 - protein, 7 aa, Homo sapiens
ATKADDK

SEQ ID NO: 145 - protein, 7 aa, Homo sapiens
MTFSENT

SEQ ID NO: 146 - protein, 7 aa, Homo sapiens
IQSSQKE

SEQ ID NO: 147 - protein, 6 aa, Homo sapiens
VYSSGN

SEQ ID NO: 148 - protein, 6 aa, Homo sapiens
IYSNGD

SEQ ID NO: 149 - protein, 6 aa, Homo sapiens
TYSSGN

SEQ ID NO: 150 - protein, 7 aa, Homo sapiens
IRSNVGE

SEQ ID NO: 151 - protein, 7 aa, Homo sapiens
IRSNMDK

SEQ ID NO: 152 - protein, 8 aa, Homo sapiens
QGSYDQQN

SEQ ID NO: 153 - protein, 4 aa, Homo sapiens
HISR

SEQ ID NO: 154 - protein, 7 aa, Homo sapiens
IRSNERE

SEQ ID NO: 155 - protein, 6 aa, Homo sapiens
SSENQE

SEQ ID NO: 156 - protein, 8 aa, Homo sapiens
RNSFDEQN

SEQ ID NO: 157 - protein, 7 aa, Homo sapiens
LYSAGEE

SEQ ID NO: 158 - protein, 7 aa, Homo sapiens
IQSSQRE

SEQ ID NO: 159 - protein, 5 aa, Homo sapiens
IPSGT

SEQ ID NO: 160 - protein, 7 aa, Homo sapiens
IRPDVSE

SEQ ID NO: 161 - protein, 7 aa, Homo sapiens
MTLNGDE

SEQ ID NO: 162 - protein, 7 aa, Homo sapiens
LVKSGEV

SEQ ID NO: 163 - protein, 5 aa, Homo sapiens
GLKNN

SEQ ID NO: 164 - protein, 5 aa, Homo sapiens
GLTSN

SEQ ID NO: 165 - protein, 7 aa, Homo sapiens
VVTGGEV

SEQ ID NO: 166 - protein, 7 aa, Homo sapiens
ISSIKDK

SEQ ID NO: 167 - protein, 7 aa, Homo sapiens
LLKGGEQ

SEQ ID NO: 168 - protein, 7 aa, Homo sapiens
LQKGGEE

SEQ ID NO: 169 - protein, 7 aa, Homo sapiens
LYKAGEL

SEQ ID NO: 170 - protein, 7 aa, Homo sapiens
LTSSGIE

SEQ ID NO: 171 - protein, 8 aa, Homo sapiens
QEAYKQQN

SEQ ID NO: 172 - protein, 8 aa, Homo sapiens
QEAYKQQN

SEQ ID NO: 173 - protein, 7 aa, Homo sapiens
LLSNGAV

SEQ ID NO: 174 - protein, 4 aa, Homo sapiens
ETME

SEQ ID NO: 175 - protein, 5 aa, Homo sapiens
LSSGK

SEQ ID NO: 176 - protein, 6 aa, Homo sapiens
FYNNEI

SEQ ID NO: 177 - protein, 6 aa, Homo sapiens
YNNKEL

SEQ ID NO: 178 - protein, 6 aa, Homo sapiens
YSYEKL

SEQ ID NO: 179 - protein, 6 aa, Homo sapiens
YNFKEQ

SEQ ID NO: 180 - protein, 6 aa, Homo sapiens
YSLEER

SEQ ID NO: 181 - protein, 6 aa, Homo sapiens
YFSETQ

SEQ ID NO: 182 - protein, 6 aa, Homo sapiens
YYREEE

SEQ ID NO: 183 - protein, 6 aa, Homo sapiens
YYEKEE

SEQ ID NO: 184 - protein, 6 aa, Homo sapiens
YYEEEE

SEQ ID NO: 185 - protein, 6 aa, Homo sapiens
YDEGEE

SEQ ID NO: 186 - protein, 6 aa, Homo sapiens
SASEGT

SEQ ID NO: 187 - protein, 6 aa, Homo sapiens
SVGEGT

SEQ ID NO: 188 - protein, 6 aa, Homo sapiens
SVGEGT

SEQ ID NO: 189 - protein, 6 aa, Homo sapiens
SNTAGT

SEQ ID NO: 190 - protein, 6 aa, Homo sapiens
SVGAGI

SEQ ID NO: 191 - protein, 6 aa, Homo sapiens
SVGAGI

SEQ ID NO: 192 - protein, 6 aa, Homo sapiens
SAAAGT

SEQ ID NO: 193 - protein, 6 aa, Homo sapiens
SVAAGI

SEQ ID NO: 194 - protein, 6 aa, Homo sapiens
FQGNSA

SEQ ID NO: 195 - protein, 6 aa, Homo sapiens
FQGTGA

SEQ ID NO: 196 - protein, 6 aa, Homo sapiens
SQSDAQ

SEQ ID NO: 197 - protein, 6 aa, Homo sapiens
FNYEAQ

SEQ ID NO: 198 - protein, 6 aa, Homo sapiens
FNYEAQ

SEQ ID NO: 199 - protein, 6 aa, Homo sapiens
FQNEAQ

SEQ ID NO: 200 - protein, 6 aa, Homo sapiens
FQNEAQ

SEQ ID NO: 201 - protein, 6 aa, Homo sapiens
YYNGEE

SEQ ID NO: 202 - protein, 6 aa, Homo sapiens
SYGVQD

SEQ ID NO: 203 - protein, 6 aa, Homo sapiens
SAAADI

SEQ ID NO: 204 - protein, 6 aa, Homo sapiens
SYGVKD

SEQ ID NO: 205 - protein, 6 aa, Homo sapiens
FQDESV

SEQ ID NO: 206 - protein, 6 aa, Homo sapiens
FQNNGV

SEQ ID NO: 207 - protein, 6 aa, Homo sapiens
YENEEA

SEQ ID NO: 208 - protein, 6 aa, Homo sapiens
FCSWTL

SEQ ID NO: 209 - protein, 5 aa, Homo sapiens
FRSSI

SEQ ID NO: 210 - protein, 6 aa, Homo sapiens
FNNNVP

SEQ ID NO: 211 - protein, 6 aa, Homo sapiens
FNNNVP

SEQ ID NO: 212 - protein, 6 aa, Homo sapiens
FRNRAP

SEQ ID NO: 213 - protein, 6 aa, Homo sapiens
FYEKMQ

SEQ ID NO: 214 - protein, 6 aa, Homo sapiens
FVKESK

SEQ ID NO: 215 - protein, 6 aa, Homo sapiens
FVKESK

SEQ ID NO: 216 - protein, 6 aa, Homo sapiens
FQNENV

SEQ ID NO: 217 - protein, 6 aa, Homo sapiens
LQKENI

SEQ ID NO: 218 - protein, 6 aa, Homo sapiens
SQIVND

SEQ ID NO: 219 - protein, 7 aa, Homo sapiens
SNEGSKA

SEQ ID NO: 220 - protein, 6 aa, Homo sapiens
SFDVKD

SEQ ID NO: 221 - protein, 6 aa, Homo sapiens
SYGVNS

SEQ ID NO: 222 - protein, 6 aa, Homo sapiens
SMNVEV

SEQ ID NO: 223 - protein, 6 aa, Homo sapiens
SYDVKM

SEQ ID NO: 224 - protein, 5 aa, Homo sapiens
SVGIG

SEQ ID NO: 225 - protein, 5 aa, synthetic construct
  REGION 1..5: note = Linker
GGGGS

SEQ ID NO: 226 - protein, 18 aa, synthetic construct
  REGION 1..18: note = linker
KESGSVSSEQLAQFRSLD

SEQ ID NO: 227 - protein, 14 aa, synthetic construct
  REGION 1..14: note = linker
EGKSSGSGSESKST

SEQ ID NO: 228 - protein, 12 aa, synthetic construct
  REGION 1..12: note = linker
GSAGSAAGSGEF

SEQ ID NO: 229 - protein, 9 aa, synthetic construct
  REGION 1..9: note = SLL peptide
SLLQHLIGL

SEQ ID NO: 230 - protein, 10 aa, synthetic construct
  REGION 1..10: note = ALY peptide
ALYVDSLFFL

SEQ ID NO: 231 - protein, 10 aa, synthetic construct
  REGION 1..10: note = ELA peptide
ELAGIGILTV

(19) United States
(12) Patent Application Publication
(10) Pub. No.: US 2024/0083967 A1
(43) Pub. Date: Mar. 14, 2024

(54) T CELL RECEPTORS AND USES THEREOF

(71) Applicant: MEDIGENE IMMUNOTHERAPIES GMBH, PLANEGG-MARTINSRIED (DE)

(72) Inventors: Christian ELLINGER, Munich (DE); Carina WEHNER, Munich (DE); Manon WEIS, Sankt Wolfgang (DE); Susanne WILDE, Germering (DE); Dolores SCHENDEL, Munich (DE)

(73) Assignee: MEDIGENE IMMUNOTHERAPIES GMBH, PLANEGG-MARTINSRIED (DE)

(21) Appl. No.: 18/236,327

(22) Filed: Aug. 21, 2023

Related U.S. Application Data
(62) Division of application No. 16/309,902, filed on Dec. 13, 2018, now Pat. No. 11,732,020.

(30) Foreign Application Priority Data
Jun. 17, 2016 (LU) 93112
Mar. 10, 2017 (EP) 17160260.0

(51) Int. Cl. C07K 14/725; A61K 35/17; A61K 39/00; A61P 35/00; C07K 14/47; C07K 14/74; C07K 16/30

(52) U.S. Cl. CPC C07K 14/7051; A61K 35/17; A61K 39/00119; A61P 35/00; C07K 14/4748; C07K 14/70539; C07K 16/3053; A61K 2039/5154; C07K 2317/34; C07K 2317/73

(57) ABSTRACT

The present invention relates to the field of biotechnology. Specifically, the invention provides antigen-specific T-cell receptors (TCRs). Further, the invention encompasses polynucleotides encoding the same and vectors comprising said polynucleotides. Host cells comprising the molecules of the invention are also provided. Moreover, the invention provides means and methods for diagnostics and therapy, in particular of cancer.

### CROSS-REFERENCE TO RELATED APPLICATIONS

The present invention is a divisional of U.S. patent application Ser. No. 16/309,902, filed Dec. 13, 2018, which was filed under 35 U.S.C. § 371 as the U.S. national phase of International Application No. PCT/EP2017/064729, filed Jun. 16, 2017, which designated the United States, and which claims the right of priority of European Patent Application No. 17160260.0 filed Mar. 10, 2017, and of Luxembourg Patent Application No. 93112 filed Jun. 17, 2016. The entire disclosures of the above-identified priority applications are hereby fully incorporated herein by reference.

### SEQUENCE LISTING

The application contains a Sequence Listing which has been submitted electronically in .XML format and is hereby incorporated by reference in its entirety. Said .XML copy, created on Oct. 6, 2023, is named “SCH-4700-DIV1.xml” and is 222 kilobytes in size. The sequence listing contained in this .XML file is part of the specification and is hereby incorporated by reference herein in its entirety.

### BACKGROUND

T lymphocytes (or T cells) which form part of the cell mediated immune system play a major role in the eradication of pathogens. T cells develop in the thymus and express T cell receptor molecules on their surface that allow the recognition of peptides presented on major histocompatibility complex (MHC) molecules which are expressed on nucleated cells (antigen presentation). Antigens of pathogens, i.e. foreign antigens presented by MHC molecules will elicit a powerful T cell response whereas self-antigens usually do not lead to a T cell response due to a negative selection of self-antigen specific T cells in the thymus during the development of such T cells. The immune system can thus discriminate between nucleated cells presenting foreign- or self-antigens and specifically target and eradicate infected cells via potent cytokine release and cellular cytotoxicity mechanisms of the T cells.

The power of the immune system has been recognized as a promising tool for future cancer therapies. In the last decade, research has begun to exploit the unique properties of T cells by using adoptive cell transfer (ACT), which involves the administration of donor-derived lymphocytes, expanded ex vivo. ACT is an attractive concept for the treatment of cancer because it does not require immune-competence of patients, and the specificity of transferred lymphocytes can be targeted against non-mutated and thus poorly immunogenic tumor antigens that typically fail to effectively trigger autologous T cell responses. Although ACT has been shown to be a promising treatment for various types of cancer, its broad application as clinical treatment has been hampered by the need for custom isolation and characterization of tumor-specific T cells from each patient—a process that can be not only difficult and time-consuming but also often fails to yield high-avidity T cells (Xue et al. Clin Exp Immunol. 2005 February; 139(2): 167-172; Schmitt et al., Hum Gene Ther. 2009 November; 20(11): 1240-1248.

The genetic transfer of tumor antigen-specific T-cell receptors (TCRs) into primary T cells can overcome some of the current limitations of ACT, as it allows for the rapid generation of tumor-reactive T lymphocytes with defined antigen specificity even in immunocompromised patients. However, the identification of suitable T cell clones bearing TCRs that specifically recognize tumor antigens and exhibit the desired anti-tumor effects in vivo is still the topic of ongoing research. Considering that in 2012 about 14.1 million new cases of cancer occurred globally and that cancer currently is the cause of about 14.6% of all human deaths worldwide, novel and efficient treatment options are urgently needed. It is the object of the present invention to comply with the needs set out above.

### SUMMARY

The present invention provides antigen-specific T cell receptors as well as nucleic acids, vectors, host cells comprising the same; and various uses and applications thereof.

In a first aspect, the invention relates to a T-cell receptor (TCR) comprising (i) a complementarity determining region 3 (CDR3) of the TCR alpha chain variable region comprising or consisting of the amino acid sequence of CAVHSTAQAGTALIF (SEQ ID NO: 1) or an amino acid sequence having at least 80% identity to SEQ ID NO:1, more preferably at least 85% identity, more preferably 90% or 95% and/or (ii) a CDR3 of the TCR beta chain variable region comprising or consisting of the amino acid sequence of CASSTHRGQTNYGYTF (SEQ ID NO. 2) or an amino acid sequence having at least 80% identity to SEQ ID NO: 2, more preferably at least 85% identity, more preferably 90% or 95% identity.

The TCRs provided herein are capable of binding to an epitope comprised within the amino acid sequence of X1LX2GLDX3LL (SEQ ID NO: 31) or its HLA-A*02 bound form, preferably to the epitope comprised within the amino acid sequence of VLDGLDVLL (SEQ ID NO: 32) or its MHC-bound form. The aforementioned amino acid sequence corresponds to amino acid positions 100 to 108 of PRAME (preferentially expressed antigen in melanoma) which is thought to be expressed by a multitude of different cancers.

TCRs, according to the invention, may for instance comprise (i) a TCR alpha chain variable region comprising or consisting of the amino acid sequence depicted in SEQ ID NO: 15, and/or (ii) a TCR beta chain variable region comprising or consisting of the amino acid sequence depicted in SEQ ID NO: 16. TCRs of the invention may also comprise a constant region in the TCR alpha and/or the TCR beta chain.

In particular, the TCRs provided herein may comprise (i) a TCR alpha chain comprising or consisting of an amino acid sequence selected from any one of SEQ ID NOs: 7, 9, 11 or 13 or an amino acid sequence having at least 80% identity, more preferably at least 85% identity, more preferably 90% or 95% to SEQ ID NO: 7, 9, 11 or 13; and/or (ii) a TCR beta-chain comprising or consisting of an amino acid sequence selected from any one of SEQ ID NOs: 8, 10, 12 or 14 or an amino acid sequence having at least 80% identity, more preferably at least 85% identity, more preferably 90% or 95% to SEQ ID NO: 8, 10, 12 or 14.

TCRs of the invention can have a variety of forms, e.g. the TCR can be a native TCR, a TCR variant, a TCR fragment, or a TCR construct. Heterodimers and multimers comprising TCR alpha and beta chains covalently linked to each other are envisaged herein as well as TCR constructs comprising one or more fusion components. A useful TCR construct comprises for instance a TCR alpha chain, a TCR beta chain (both covalently linked to each other) and an antibody or antibody fragment, such as a svFv, which is directed against an antigen or epitope on the surface of lymphocytes (e.g. CD3, CD28, CD5, CD16 or CD56) and is fused to the TCR chains via a linker.

Other useful moieties that can be covalently linked to the inventive TCRs comprise various labels. The TCRs of the invention can also be provided in soluble form.

Further, the invention provides a nucleic acid encoding any of the TCRs provided herein, said nucleic acid for instance comprising or consisting of the nucleic acid sequence of any one of SEQ ID NOs: 23, 24, 25, 26, 27, 28, 29 or 30.

Further provided herein is a vector, comprising the nucleic acid according to the invention. Exemplary vectors include viral vectors, e.g. lentiviral or gamma-retroviral vectors.

Host cells comprising the TCR, the nucleic acid, or the vector of the invention are also provided herein. Useful host cells include lymphocytes such as cytotoxic T lymphocytes (CTLs), CD8+ T cells, CD4+ T cells, natural killer (NK) cells, natural killer T (NKT) cells, gamma/delta-T-cells.

Moreover, the invention provides a method for obtaining the TCR of the invention.

A pharmaceutical or diagnostic composition comprising the TCR, nucleic acid, vector and/or host cell of the invention is also provided herein. Use of the inventive TCR, nucleic acid, vector, host cell and/or pharmaceutical composition as a medicament or diagnostic agent, and in particular detection, diagnosis, prognosis, prevention and/or treatment of cancer is also envisaged. A useful way of preventing or treating cancer includes the following steps: (a) providing one or more of the TCR, nucleic acid, vector, host cell and/or pharmaceutical composition disclosed herein; and (b) administering one or more of the aforementioned to a subject in need thereof. The invention also envisages the following: (a) providing a sample of a subject, said sample comprising lymphocytes; (b) providing one or more of the TCR, nucleic acid, vector, host cell and/or pharmaceutical composition disclosed herein, and (c) introducing the same into the lymphocytes obtained in step (a) and, thereby, obtaining modified lymphocytes, (d) administering the modified lymphocytes of step (c) to a subject or patient in need thereof.

The invention further relates to an in vitro method of detecting the presence of a cancer in a subject in vitro, comprising (a) providing a sample of a subject, said sample comprising one or more cells; (b) contacting said sample with the TCR, nucleic acid, vector or host cell of the invention, thereby forming a complex, and (c) detecting the complex, wherein detection of the complex is indicative of the presence of the cancer in the subject.

### DESCRIPTION OF THE FIGURES

FIG. 1 shows a schematic overview of the priming approach using mature dendritic cells. PRAME-transfected mature dendritic cells were used to de-novo induce PRAME-specific CD8 T cells within the repertoire of the autologous healthy donor.

FIG. 2 shows multiplex cytokine secretion analysis (IFN-gamma, IL-2, TNF-alpha, IL-5, IL-10, IL-6, IL12p70, IL-4, IL-1beta) of PRAME₁₀₀₋₁₀₈-specific T cell clone T4.8-1-29 co-cultured with peptide-loaded T2 cells (PRAME₁₀₀₋₁₀₈ “VLD peptide” or PRAME₃₀₀₋₃₀₉ “ALY peptide” as negative control, n.d.=not detected). T4.8-1-29 is characterized by having a CDR3 of its TCR alpha chain variable region as shown in SEQ ID NO: 1 and/or by having a CDR3 of its TCR beta chain variable region as shown in SEQ ID NO: 2.

FIG. 3 shows IFN-gamma release from PRAME₁₀₀₋₁₀₈-specific T cell clone T4.8-1-29 co-cultured with various human tumor cell lines expressing HLA-A*02:01, wherein, as indicated in the legend of FIG. 3, some of the tumor cell lines express PRAME (green bars). Tumor cell lines not expressing PRAME serve as negative control (red bars). Positive control: T2 cells loaded with “VLD peptide” (black bar), background of the T cells without stimulation is indicated by white bars (“n.d.=not detected”).

FIG. 4 shows IFN-gamma release of PRAME₁₀₀₋₁₀₈-specific T cell clone T4.8-1-29 co-cultured with T2 cells loaded with titrated amounts of PRAME₁₀₀₋₁₀₈-peptide. The dotted line indicates the peptide concentration leading to half-maximal IFN-gamma secretion between 10⁻⁹-10⁻¹⁰ mol/L [M] as a measure for the functional avidity of the tested T cell clone.

FIG. 5 To prove pairing and functionality of the transgenic TCR, specific IFN-gamma release of PRAME₁₀₀₋₁₀₈-specific TCR T4.8-1-29-transfected recipient CD8+ T (Recipient T cell clone+T4.8-1-29 ivtRNA) cells in co-culture with PRAME₁₀₀₋₁₀₈ (VLD) peptide-loaded T2 cells or T2 cells loaded with irrelevant peptide was measured by standard ELISA.

FIG. 6 shows specific IFN-gamma release from PRAME₁₀₀₋₁₀₈-specific CD8+ enriched PBMC engineered to express the PRAME₁₀₀₋₁₀₈-specific TCR T4.8-1-29 in co-culture with self-peptide-loaded (in total 131 ubiquitous self-peptides binding to HLA-A*02:01 encoded molecules) T2 cells measured using standard ELISA.

FIG. 7 shows lysis of T2 cells loaded either with PRAME₁₀₀₋₁₀₈-peptide (“VLD-Peptide”) or irrelevant peptide SLLQHLIGL (SEQ ID NO: 229) (“SLL-Peptide”) by CD8+ enriched PBMC engineered to express the PRAME₁₀₀₋₁₀₈-specific TCR T4.8-1-29.

FIG. 8 shows lysis of PRAME₁₀₀₋₁₀₈-expressing target cells by human PBMC expressing the PRAME₁₀₀₋₁₀₈-specific TCR T4.8-1-29. (A) lysis of HLA-A*02:01-transfected, endogenously PRAME positive human K562 tumor cells, additionally loaded with PRAME₁₀₀₋₁₀₈-peptide (“VLD peptide”). (B) HLA-A*02 negative, endogenously PRAME positive human K562 cells additionally transfected with ivtRNA coding for PRAME as negative control are not lysed. (C) shows lysis of HLA-A*02-transfected, endogenously PRAME positive human K562 cells additionally transfected with ivtRNA coding for PRAME. (D) shows lysis of HLA-A*02-transfected, endogenously PRAME positive human K562.

FIG. 9 shows the amino acid sequences of a useful example of a T cell receptor alpha and beta chain (SEQ ID NOs 11 and 12) and the amino acid sequence of human PRAME (SEQ ID NO. 33). In the alpha and beta chain, CDR1 and CDR2 sequences are underlined, CDR3 sequences are in grey and bold, variable regions in regular font, constant region in italics.

FIG. 10 shows recognition of different HLA-A*02 and PRAME positive tumor cell lines by CD8+ enriched PBMC expressing TCR T4.8-1-29HLA-A*02 as indicated by activation-induced IFN-gamma release and measured by standard ELISA.

FIG. 11 shows non-transduced PBMC of a healthy donor and the same PBMC of the healthy donor transduced with a plasmid containing the TCT T4.8-1-29 construct described herein.

FIG. 12 shows functional T cell avidity for the PRAME₁₀₀₋₁₀₈ (VLD) peptide as measured by detection of IFN-gamma secretion after co-culturing either the T cell clone T4.8-1-29 (dotted curve) or effector PBMC transduced with T4.8-1-29 (solid curve) with peptide-loaded T2 cells.

FIG. 13 shows the analysis of antigen specificity of T4.8-1-29-transduced effector PBMC and untransduced control PBMC. The tumor cell lines OPM-2 and U937 (HLA-A2-negative and PRAME-negative) were tested either unmodified, or transfected with ivtRNA encoding HLA-A2

FIG. 14 shows the analysis of antigen specificity, T4.8-1-29-transduced effector PBMC and untransduced control PBMC were cocultured with different target cell lines. The tumor cell lines K562 (HLA-A2-negative and PRAME-positive) were tested as well as K562 A2 and Mel 624.38 (HLA-A-positive and PRAME-positive) and 647-V (HLA-A2-positive and PRAME-negative).

FIG. 15 shows the analysis of cytotoxic activity of T4.8-1-29-transduced effectors against tumor cells using the IncuCyte ZOOM®—Live Cell Analysis System (Essenbiosciences), a microscope-based system that allows live imaging of cells.

FIG. 16 shows the analysis of the safety profile of T4.8-1-29-expressing PBMC

### DETAILED DESCRIPTION

The present inventors have identified T cell clones that are capable of specifically recognizing cells expressing the tumor-associated antigen (TAA) PRAME; and that exhibit advantageous effector functions such as cytokine production and cytolysis of target cells. Said T cell clones and their T cell receptors are therefore promising tools for highly specific and effective cancer treatment. The identified PRAME-specific TCRs are thus suitable for adoptive T cell therapy of cancer. The identification of a TCR that is capable of binding to PRAME in a highly specific manner thus allows for “arming” T cells ex vivo and re-introducing them into the donor where they can effectively recognize and specifically eliminate PRAME expressing cancer cells. Moreover, the antigen binding regions of the novel TCR provided herein can be used to design soluble constructs comprising further functional moieties (such as drugs, labels or further binding domains attracting other immune cells) that are readily available for direct administration.

### Variable Region

### CDR3 Domains

In a first aspect, the present invention thus relates to a T-cell receptor (TCR) comprising (i) a T cell receptor alpha-chain CDR3 comprising or consisting of the sequence of CAVHSTAQAGTALIF (SEQ ID NO: 1) and/or (ii) a T-cell receptor beta-chain CDR3 comprising or consisting of the amino acid sequence of CASSTHRGQTNYGYTF (SEQ ID NO. 2)

Further envisaged herein are TCR sequence variants comprising a CDR3 alpha comprising or consisting of an amino acid sequence having at least 80% identity, more preferably at least 85% identity, more preferably 90% or 95% to SEQ ID NO: 1 and/or CDR3beta comprising or consisting of an amino acid sequence having at least 80% identity, more preferably at least 85% identity, more preferably 90% or 95% to SEQ ID NO: 2; provided that the TCR retains the advantageous capabilities of the TCR evaluated in the appended examples, i.e. is capable of binding to the antigenic target specified herein.

The term “T cell receptor” or “TCR” as used herein includes native TCRs as well as TCR variants, fragments and constructs. The term thus includes heterodimers comprising TCR alpha and beta chains as well as multimers and single chain constructs; optionally comprising further domains and/or moieties.

In its native form, the TCR exists as a complex of several proteins on the surface of T cells. The T cell receptor is composed of two (separate) protein chains, which are produced from the independent T cell receptor alpha and beta (TCR α and TCR β) genes and are called alpha (α-) and beta (β-) chains. Each chain of the TCR possesses one N-terminal immunoglobulin-like (Ig)-variable (V) domain/region, one Ig-constant-like (C) domain/region, a transmembrane/cell membrane-spanning region anchoring the chain in the plasma membrane, and a short cytoplasmic tail at the C-terminal end.

Antigen specificity is conferred by the variable regions of the alpha and beta chain. Both variable domains of the TCR alpha chain and beta chain comprise three hypervariable or complementarity determining regions (CDR1alpha/beta, CDR2alpha/beta and CDR3 alpha/beta) surrounded by framework (FR) regions. CDR3 is the prime determinant of antigen recognition and specificity (i.e. the ability to recognize and interact with a specific antigen), whereas CDR1 and CDR2 mainly interact with the MHC molecule presenting the antigenic peptide.

Native TCRs recognize antigenic peptides bound to (“presented/displayed on”) major histocompatibility complex (MHC) molecules at the surface of an antigen presenting cell. An antigenic peptide presented on a MHC molecule is also referred to as a “peptide:MHC complex” herein. There are two different classes of MHC molecules: MHC I and MHC II, which present peptides from different cellular compartments. MHC class I molecules are expressed on the surface of all nucleated cells throughout the human body and display peptide or protein fragments from intracellular compartments to cytotoxic T cells. In humans, the MHC is also called the human leukocyte antigen (HLA). There are three major types of MHC class I: HLA-A, HLA-B and HLA-C. Once a TCR binds to its specific peptide:MHC complex, the T cell is activated and exerts biological effector functions.

The TCRs provided herein are advantageously capable of (specifically) recognizing PRAME, in particular PRAME₁₀₀₋₁₀₈ in its MHC bound form as will be discussed below in detail. An antigenic peptide is said to be present in its “MHC bound form” when it forms a complex with an MHC molecule (which may be present on the surface of an antigen presenting cell such as a dendritic cell or a tumor cell, or it may be immobilized by for example coating to a bead or plate.)

### CDR1 and CDR2 Domains

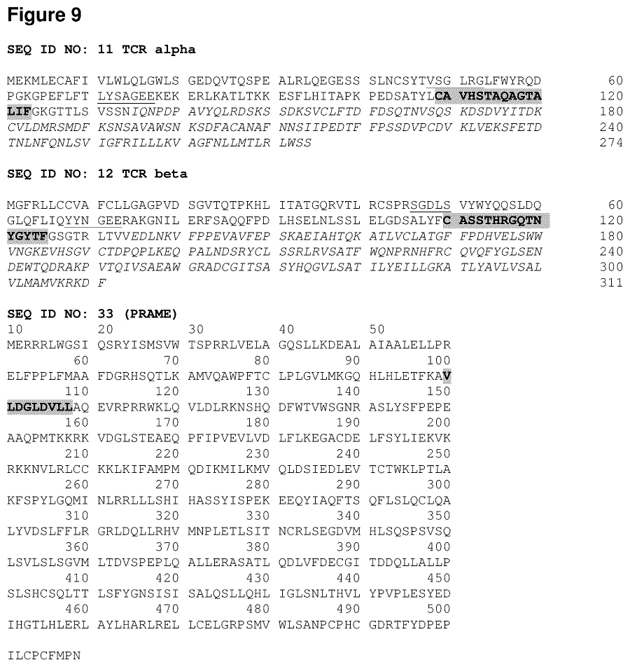

As set out previously, the TCRs of the invention are particularly envisaged to recognize their antigenic target PRAME₁₀₀₋₁₀₈ when being presented on an MHC molecule, specifically an MHC-I molecule, and in particular HLA-A, preferably HLA-A*02 and specifically HLA-A2 molecules encoded by the allele HLA-A*02:01 (the T cell or TCR is said to be “restricted” to a particular MHC molecule). It is also conceivable that the TCRs of the invention recognize their antigenic target presented on other HLA-A*02 alleles. As noted previously, CDR1 and CDR2 of the TCR alpha and beta chains are thought to be mainly involved in MHC recognition. There is a limited “pool” of CDR1 and CDR2 sequences known to be involved in HLA-A*02-restricted antigen recognition, and it is envisaged that the CDR3 domains of the present invention can in principle be combined with any of the CDR1 and CDR2 domains depicted in SEQ ID NO: 34-224, provided that the TCR retains its ability to recognize its antigenic target, preferably in its HLA-A*02 bound form, to a similar, the same or even a higher extent as the TCR evaluated in the appended examples. Useful examples of CDR1 and CDR2 domains include the CDR1 alpha comprising or consisting of the sequence VSGLRG as depicted in SEQ ID NO: 5, the CDR2 alpha comprising or consisting of the sequence LYSAGEE as depicted in SEQ ID NO: 3, the CDR1 beta comprising or consisting of the sequence SGDLS as depicted in SEQ ID NO: 6, and the CDR2 beta comprising or consisting of the sequence YYNGEE as depicted in SEQ ID NO: 4. Said CDR sequences are also shown in FIG. 9.

In accordance with the foregoing, the present invention inter alia provides TCRs comprising two polypeptide chains, each of which comprises a human variable region comprising at least one complementarity determining region (i.e. in particular CDR3, and preferably a CDR1, and/or CDR2) of a TCR. A TCR with particular advantageous properties (as shown in the appended examples) comprises a first polypeptide chain comprising a CDR1 comprising or consisting of the amino acid sequence of SEQ ID NO: 5 (CDR1 alpha), a CDR2 comprising or consisting of the amino acid sequence of SEQ ID NO: 3 (CDR2 alpha), and a CDR3 comprising or consisting of the amino acid sequence of SEQ ID NO: 1 (CDR3 alpha), and second polypeptide chain comprising a CDR1 comprising or consisting of the amino acid sequence of SEQ ID NO: 6 (CDR1 beta), a CDR2 comprising or consisting of the amino acid sequence of SEQ ID NO: 4 (CDR2 beta), and a CDR3 comprising or consisting of the amino acid sequence of SEQ ID NO: 2 (CDR3 beta).

### Complete Variable Regions

The present invention further provides a TCR comprising a TCR alpha chain variable region comprising or consisting of an amino acid sequence as depicted in SEQ ID NO: 15 and/or a TCR beta chain variable region comprising or consisting of an amino acid sequence as depicted in SEQ ID NO: 16. Said alpha and beta chain sequences are also shown in FIG. 9 (normal font).

TCR sequence variants comprising alpha chain variable regions comprising an amino acid sequence having at least 80% identity, more preferably at least 85% identity, more preferably 90% or 95% to SEQ ID NO: 15 and/or a TCR beta chain variable region comprising or consisting of an amino acid sequence having at least 80% identity, more preferably at least 85% identity, more preferably 90% or 95% to SEQ ID NO: 16 are also envisaged herein; provided that the TCR retains the advantageous capabilities of the TCR evaluated in the appended examples, i.e. is capable of binding to the antigenic target specified herein.

### Constant Region

The TCR may further comprise a constant (C) region in its alpha and/or beta chain. The constant region can be a human constant region or derived from another species, yielding a “chimeric” TCR. For instance, human alpha and/or beta chains can be replaced by their murine counterparts (“murinization”) which has been found to enhance surface expression of human TCRs by supporting preferential pairing of the TCR alpha and beta chains, and a more stable association with the CD3 co-receptor. Suitable constant regions of the alpha chain can for instance be selected from SEQ ID NOs: 17 (human), 19 (minimal murinized) and 21 (murine). Suitable constant regions of the beta chain can be selected from SEQ ID NOs: 18 (human), 20 (minimal murinized) and 22 (murine). Instead of replacing complete human constant regions by their murine counterparts, it is also possible to exchange only some amino acids in the human constant regions for the corresponding amino acids of the murine constant region (“minimal murinization”), as further explained in the section “TCR sequence variants” herein.

### Alpha and Beta Chains

Useful examples of TCRs of the invention include those comprising an alpha chain comprising or consisting of an amino acid sequence as depicted in SEQ ID NO: 7, 9, 11 or 13 and a beta chain comprising or consisting of an amino acid sequence as depicted in SEQ ID NO: 8, 10, 12 or 14. The present invention thus provides, inter alia, a TCR comprising or consisting of an alpha chain comprising or consisting of an amino acid sequence as depicted in SEQ ID NO: 7 and a beta chain comprising or consisting of an amino acid sequence as depicted in SEQ ID NO: 8; a TCR comprising or consisting of an alpha chain comprising or consisting of an amino acid sequence as depicted in SEQ ID NO: 9 and a beta chain comprising or consisting of an amino acid sequence as depicted in SEQ ID NO: 10; a TCR comprising or consisting of an alpha chain comprising or consisting of an amino acid sequence as depicted in SEQ ID NO: 11 and a beta chain comprising or consisting of an amino acid sequence as depicted in SEQ ID NO: 12 (both also shown in FIG. 9); and a TCR comprising or consisting of an alpha chain comprising or consisting of an amino acid sequence as depicted in SEQ ID NO: 13 and a beta chain comprising or consisting of an amino acid sequence as depicted in SEQ ID NO: 14.

TCR sequence variants comprising alpha chains comprising an amino acid sequence having at least 80% identity, more preferably at least 85% identity, more preferably 90% or 95% to SEQ ID NO: 7, 9, 11 or 13 and/or a TCR beta chain comprising or consisting of an amino acid sequence having at least 80% identity, more preferably at least 85% identity, more preferably 90% or 95% to SEQ ID NO: 8, 10, 12 or 14 are also envisaged herein; provided that the TCR retains the advantageous capabilities of the TCR evaluated in the appended examples, i.e. is capable of binding to the antigenic target specified herein.

### Antigenic Target

The TCRs provided herein are advantageously capable of binding to (human) PRAME. PRAME (Melanoma antigen preferentially expressed in tumors, Uniprot Acc. No. P78395), also referred to as MAPE (melanoma antigen preferentially expressed in tumors) and OIP4 (OPA-interacting protein 4), has been reported a cancer-testis antigen (CTA) with unknown function.

In particular, the present invention provides TCRs that are capable of (specifically) binding to an epitope comprised within an amino acid sequence corresponding to amino acid positions 100-108 of the PRAME amino acid sequence as depicted in SEQ ID NO: 33 (FIG. 9) in bold, The PRAME peptide consisting of the amino acid sequence as depicted in SEQ ID NO: 32 is also referred to as PRAME₁₀₀₋₁₀₈ or the “antigenic target” or “VLD peptide” herein. As set out elsewhere herein, the TCR- of the invention will preferably recognize PRAME₁₀₀₋₁₀₈ when bound by MHC, in particular HLA-A*02.

The term “position” when used in accordance with the disclosure means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids, but is rather to be viewed in the context of the circumjacent portion of the sequence. Accordingly, the position of a given amino acid or nucleotide in accordance with the disclosure may vary due to deletion or addition of amino acids or nucleotides elsewhere in the sequence. Thus, when a position is referred to as a “corresponding position” in accordance with the disclosure it is understood that nucleotides/amino acids may differ in terms of the specified numeral but may still have similar neighboring nucleotides/amino acids. In order to determine whether an amino acid residue (or nucleotide) in a given sequence corresponds to a certain position in the amino acid sequence of a “parent” amino acid/nucleotide sequence, the skilled person can use means and methods well-known in the art, e.g., sequence alignments, either manually or by using computer programs such as exemplified herein.

The term “epitope” in general refers to a site on an antigen, typically a (poly-) peptide, which a binding domain recognizes. The term “binding domain” in its broadest sense refers to an “antigen binding site”, i.e. characterizes a domain of a molecule which binds/interacts with a specific epitope on an antigenic target. An antigenic target may comprise a single epitope, but typically comprises at least two epitopes, and can include any number of epitopes depending on the size, conformation, and type of antigen. The term “epitope” in general encompasses linear epitopes and conformational epitopes. Linear epitopes are contiguous epitopes comprised in the amino acid primary sequence and typically include at least 2 amino acids or more. Conformational epitopes are formed by non-contiguous amino acids juxtaposed by folding of the target antigen, and in particular target (poly-) peptide.

In the context of the present invention the term “binding domain” in particular refers to the variable region of the TCR alpha and/or beta chain and specifically the CDR3alpha and CDR3beta of the TCR.

The present inventors have found that the minimal amino acid sequence recognized by the TCRs of the invention corresponds to the amino acid sequence X1LX2GLDX3LL (SEQ ID NO: 31), with X being selected from any amino acid. Specifically, the inventive TCRs has been shown to (specifically) recognize the amino acid sequence comprising or consisting of the amino acid sequence VLDGLDVLL (SEQ ID NO: 32), as shown in the appended examples. The TCRs of the invention are thus capable of binding to an amino acid sequence comprising or consisting of the amino acid sequence X1LX2GLDX3LL (SEQ ID NO: 31), preferably comprising or consisting of the amino acid sequence VLDGLDVLL (SEQ ID NO: 32) or its MHC bound form. For instance, it is envisaged that the recognized peptide may comprise further C amino acids located C- and/or N-terminal of the recognition motif depicted in SEQ ID NO: 31 and in particular SEQ ID NO: 32. Specifically, the TCR described herein is envisaged to recognize at least one epitope within the aforementioned amino acid sequences. The terms “binding to” and “recognizing” in all grammatical forms are used interchangeably herein. The antigenic target is particularly envisaged to be recognized by the inventive TCR when being bound by a MHC class I molecule, specifically a HLA-A molecule, and preferably a HLA-A*02 molecule, in particular a HLA-A*02:01 molecule. Said MHC molecule, in particular HLA-A and HLA-A*02 molecule, can be present on the surface of a cell, for instance a tumor cell, or on a (solid) carrier.

Preferably, the inventive TCRs specifically bind to their antigenic target. The term “specific(ally) binding” generally indicates that a TCR binds via its antigen binding site more readily to its intended antigenic target than to a random, unrelated non-target antigen. Particularly the term “specifically binds” indicates that the binding specificity of the TCR will be at least about 5-fold, preferably 10-fold, more preferably 25-fold, even more preferably 50-fold, and most preferably 100-fold or more, greater for its antigenic target than its binding specificity for a non-target antigen.

Effector host cells expressing a native TCR as described herein are envisaged to bind to their antigenic target (i.e. preferably PRAME₁₀₀₋₁₀₈ presented on HLA-A*02 by antigen presenting cells) with a high functional avidity. The term “functional avidity” refers to the capability of TCR expressing cells (in particular T-cells expressing native TCRs as described herein) to respond in vitro to a given concentration of a ligand and is thought to correlate with the in vivo effector capacity of TCR expressing cells. By definition, TCR expressing cells with high functional avidity respond in in vitro tests to very low antigen doses, while such cells of lower functional avidity require higher amounts of antigen before they mount an immune response similar to that of high-avidity TCR expressing cells. The functional avidity can be therefore considered as a quantitative determinant of the activation threshold of a TCR expressing cell. It is determined by exposing such cells in vitro to different amounts of cognate antigen. TCR expressing cells with high functional avidity respond to low antigen doses. For example, a TCR expressing cell will typically be considered to bind with “high” functional avidity to its antigenic target if it secretes at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, or 20,000 pg/mL or more) of interferon gamma (IFN-gamma) upon co-culture with antigen-negative HLA-A*02 expressing target cells loaded with a low concentration of the PRAME₁₀₀₋₁₀₈ peptide ranging from about 10⁻⁵ to about 10⁻¹¹ M (i.e., about 0.05 ng/mL to about 5 ng/mL, 0.05 ng/mL, 0.1 ng/mL, 0.5 ng/mL, 1 ng/mL, or 5 ng/mL) with a molecular weight of the PRAME₁₀₀₋₁₀₈ peptide of 956 g/mol.

Other methods to determine specific binding of the inventive TCRs include the 51 Cr-release assay described by Gertner-Dardenne et al. J Immunol 188(9): 4701-4708, CD107a/b mobilization described by Leisegang et al., Clin. Cancer Res 2010. 16: 2333-2343 and peptide:MHC multimer binding analyses described by Wilde et al., J Immunol 2012; 189:598-605.

### Variants

As noted previously, the term “TCR” encompasses TCR variants, which include TCR sequence variants, fragments and constructs. All TCR variants are envisaged to be functional variants of the inventive TCR. The term “functional variant” as used herein refers to a TCR, polypeptide, or protein having substantial or significant sequence identity or similarity to a parent TCR, its variable regions or its antigen-binding regions and shares its biological activity, i.e. its ability to specifically bind to the antigenic target for which the parent TCR of the invention has antigenic specificity to a similar, the same or even a higher extent as the TCR disclosed herein and evaluated in the appended examples.

### Sequence Variants

The term “TCR variants” includes “sequence variants” of the TCRs disclosed herein, i.e. variants substantially comprising the amino acid sequence of the inventive TCR as described above (also referred to as the “parent” TCR) but containing at least one amino acid modification (i.e. a substitution, deletion, or insertion) as compared to the “parent” TCR amino acid sequence, provided that the variant preferably retains the antigenic specificity of the inventive “patent” TCR. TCR sequence variants of the invention are typically prepared by introducing appropriate nucleotide changes into the nucleic acids encoding the “parent” TCR, or by peptide synthesis. Generally, the aforementioned amino acid modifications may be introduced into, or present in, the variable region or the constant region of the TCR, and may serve to modulate properties like binding strength and specificity, post-translational processing (e.g. glycosylation), thermodynamic stability, solubility, surface expression or TCR assembly.

As set out previously, amino acid modifications include, for example, deletions from, and/or insertions into, and/or substitutions of, residues within the amino acid sequences of the parent TCR. Exemplary insertional variants of a TCR of the invention include fusion products of said TCR and an enzyme or another functional polypeptide. Exemplary substitutional variants of a TCR of the invention are those including amino acid substitutions in variable regions or CDRs of the alpha and/or beta chain, the framework region or the constant region. Particularly envisaged herein are conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be in an acidic amino acid substituted for another acidic amino acid (e.g., Asp or Glu), an amino acid with a nonpolar side chain substituted for another amino acid with a nonpolar side chain (e.g., Ala, Gly, Val, He, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gin, Ser, Thr, Tyr, etc.), etc. that may be made, for instance, on the basis of similarity in polarity, charge, solubility, hydrophobicity, hydrophilicity, and/or the amphipathic nature of the residues involved.

### Cysteine Modification

The addition of a disulfide bond in the constant region has been reported to foster correct pairing of the TCR alpha and beta chains (Kuball J et al. Blood. 2007 Mar. 15; 109(6):2331-8.). Thus, the addition of one or more cysteine bonds in the constant region is also envisaged herein.

### Murinization

As noted previously, murinization of TCRs (i.e. exchanging the human constant regions in the alpha and beta chain by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells engineered ex vivo to express the TCRs of desired antigenic specificity, but still retaining and expressing their “original” TCRs.

Recently nine amino acids responsible for the improved expression of murinized TCRs have been identified (Sommermeyer and Uckert, J Immunol. 2010 Jun. 1; 184(11):6223-31) and it is envisaged to substitute one or all of the amino acid residues in the TCRs alpha and//or beta chain constant region for their murine counterpart residues. This technique is also referred to as “minimal murinization”, and offers the advantage of enhancing cell surface expression while, at the same time, reducing the number of “foreign” amino acid residues in the amino acid sequence and, thereby, the risk of immunogenicity.

In general, TCR sequence variants are envisaged to comprise at least one of the CDR1, CDR2, CDR3, alpha chain variable regions, beta chain variable regions, alpha chains and/or beta chains as disclosed herein, or comprising or consisting of an amino acid sequence that is at least about 80%, about 85%, about 90%, about 95%, about 96%, about 97%, about 98%, about 99%, or identical to the amino acid sequences disclosed herein, provided that said variants exhibit comparable, the same or improved binding characteristics as compared to TCR evaluated in the appended examples.

As used herein the term “sequence identity” indicates the extent to which two (nucleotide or amino acid) sequences have identical residues at the same positions in an alignment, and is often expressed as a percentage. Preferably, identity is determined over the entire length of the sequences being compared. Thus, two copies of exactly the same sequence have 100% identity, but sequences that are less highly conserved and have deletions, additions, or replacements, may have a lower degree of identity. Those skilled in the art will recognize that several algorithms are available for determining sequence identity using standard parameters, for example Blast (Altschul, et al. (1997) Nucleic Acids Res. 25:3389-3402), Blast2 (Altschul, et al. (1990) J. Mol. Biol. 215:403-410), Smith-Waterman (Smith, et al. (1981) J. Mol. Biol. 147:195-197) and ClustalW.

Accordingly, the amino acid sequences of SEQ ID Nos: 1 or 2, can for instance serve as “subject sequence” or “reference sequence”, while the amino acid sequence of a CDR3 different therefrom can serve as “query sequence”.

### Constructs and Fragments

The term “TCR” as used herein further comprises TCR constructs. The term “construct” includes proteins or polypeptides comprising at least one antigen binding domain of the inventive TCRs, but do not necessarily share the basic structure of a native TCR (i.e. variable domains incorporated into a TCR alpha chain and a TCR beta chain forming a heterodimer). TCR constructs and fragments are typically obtained by routine methods of genetic engineering and are often artificially constructed to comprise additional functional protein or polypeptide domains. In accordance with the foregoing, TCR constructs and fragments of the invention are envisaged to comprise at least one CDR3alpha and/or at least one CDR3beta as disclosed elsewhere herein. Further envisaged herein are constructs and fragments comprising at least one CDR1alpha, CDR2alpha, CDR1beta, CDR2beta, alpha chain variable region, beta chain variable region, alpha chain and/or beta chain, or combinations thereof, optionally in combination with further protein domains or moieties as exemplified herein. The TCR constructs and fragments provided herein are envisaged to be capable of specifically binding to the same antigenic target as the inventive TCRs described above and evaluated in the appended Examples.

### Multimers

The term “TCR construct” encompasses heterodimers and multimers in which at least one TCR alpha chain variable region or TCR alpha-chain and at least one TCR beta-chain variable region are covalently linked to each other. In its simplest form a multivalent TCR construct according to the invention comprises a multimer of two or three or four or more TCRs associated (e. g. covalently or otherwise linked) with one another, preferably via a linker molecule.

Suitable linker molecules include, but are not limited to, multivalent attachment molecules such as avidin, streptavidin, neutravidin and extravidin, each of which has four binding sites for biotin. Thus, biotinylated TCRs can be formed into multimers having a plurality of TCR binding sites. The number of TCRs in the multimer will depend upon the quantity of TCR in relation to the quantity of linker molecule used to make the multimers, and also on the presence or absence of any other biotinylated molecules. Exemplary multimers are dimeric, trimeric, tetrameric or pentameric or higher-order multimer TCR constructs. Multimers of the invention may also comprise further functional entities such as labels or drugs or (solid) carriers.

The term “TCR construct” also encompasses TCR molecules which are linked via a suitable linker to a spheric body, preferably a uniform bead, more preferably a polystyrene bead, most preferably a bio-compatible polystyrene bead. Such TCR constructs can also be comprised of an inventive TCR and a bead having a pre-defined fluorescence dye incorporated into the bead.

### Fusion Proteins

The term “TCR construct” also relates to fusion proteins or polypeptides comprising at least one TCR alpha chain, TCR alpha chain variable region or CDR3alpha and/or at least one TCR beta chain, TCR beta chain variable region or CDR3beta; and further one or more fusion component(s). Useful components include Fc receptors; Fc domains (derived from IgA, IgD, IgG, IgE, and IgM); cytokines (such as IL-2 or IL-15); toxins; antibodies or antigen-binding fragments thereof (such as anti-CD3, anti-CD28, anti-CD5, anti-CD 16 or anti-CD56 antibodies or antigen-binding fragments thereof); CD247 (CD3-zeta), CD28, CD137, CD134 domains; or any combinations thereof.

Exemplary antibody fragments that can be used as fusion components include fragments of full-length antibodies, such as (s)dAb, Fv, Fd, Fab, Fab′, F(ab′)2 or “r IgG” (“half antibody”); modified antibody fragments such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies, multibodies such as triabodies or tetrabodies, and single domain antibodies such as nanobodies or single variable domain antibodies comprising only one variable domain, which might be V_{H}H, V_{H} or V_{L}.

TCR constructs of the invention may be fused to one or more antibody or antibody fragments, yielding monovalent, bivalent and polyvalent/multivalent constructs and thus monospecific constructs, specifically binding to only one target antigen as well as bispecific and polyspecific/multispecific constructs, which specifically bind more than one target antigens, e.g. two, three or more, through distinct antigen binding sites.

Optionally, a linker may be introduced between the one or more of the domains or regions of the TCR construct of the invention, i.e. between the TCR alpha chain CDR3, TCR alpha chain variable region, and/or a TCR alpha chain, the TCR beta chain CDR3, TCR beta chain variable region, and/or a TCR beta chain, and/or the one or more fusion component(s) described herein. Linkers are known in the art and have been reviewed, inter alia, by Chen et al. Adv Drug Deliv Rev. 2013 Oct. 15; 65(10): 1357-1369. In general, linkers include flexible, cleavable and rigid linkers and will be selected depending on the type of construct and intended use/application. For example, for therapeutic application, non-immunogenic, flexible linkers are often preferred in order to ensure a certain degree of flexibility or interaction between the domains while reducing the risk of adverse immunogenic reactions. Such linkers are generally composed of small, nonpolar (e.g. Gly) or polar (e.g. Ser or Thr) amino acids and include “GS” linkers consisting of stretches of Gly and Ser residues. An example of the most widely used flexible linker which is also intended for use in the TCR construct of the present invention has the sequence of (Gly-Gly-Gly-Gly-Ser), (SEQ ID NO: 225) Other suitable linkers include for instance KESGSVSSEQLAQFRSLD (SEQ ID NO: 226) and EGKSSGSGSESKST (SEQ ID NO: 227) and GSAGSAAGSGEF (SEQ ID NO: 228).

Particularly useful TCR constructs envisaged in accordance with the invention are those comprising at least one TCR alpha chain, TCR alpha chain variable region or CDR3 alpha as defined herein, at least one TCR beta chain, TCR beta chain variable region or CDR3 beta as defined herein, optionally linked to each other and fused, optionally via a liker, to at least one antibody or an antibody fragment (such as a single chain antibody fragment (scFv)) directed against an antigen or epitope on the surface of lymphocytes. Useful antigenic targets recognized by the antibody or antibody fragment (e.g. scFv) include CD3, CD28, CD5, CD16 and CD56. Said construct can in general have any structure as long the “TCR portion” (i.e. TCR alpha and beta chain or variable regions or CDR3s thereof) retains its ability to recognize the antigenic target defined herein, and the “antibody portion” binds to the desired surface antigen or epitope, thereby recruiting and targeting the respective lymphocyte to the target cell. Such constructs may advantageously serve as “adapters” joining an antigen presenting cell displaying the antigenic target (such as a tumor cell) and a lymphocyte (such as a cytotoxic T cell or NK cell) together. An example of such a fusion protein is a construct engineered according to the principle of a bispecific T-cell engager (BiTE®) consisting of two single-chain variable fragments (scFvs) of different antibodies, on a single peptide chain of about 55 kilodaltons (kD). Accordingly, a TCR construct of the invention may comprise at least one TCR antigen binding domain as described herein (for instance a TCR variable alpha and variable beta chain fused to each other) linked to a scFv (or other binding domain) of the desired binding specificity, e.g. CD3 or CD56. The scFv (or other binding domain) binds to T cells such as via the CD3 receptor or to CD56 for NK cell activation, and the other to a tumor cell via an antigenic target specifically expressed on the tumor cell. Also envisaged herein are tribodies comprising at least one TCR antigen binding domain as described herein, an scFv (or other binding domain) and a further domain e.g. for targeting the construct to a site of action within the body (e.g. an Fc domain).

### Isolated Form

The TCRs of the invention may be provided in “isolated” or “substantially pure” form. “Isolated” or “substantially pure” when used herein means that the TCRs have been identified separated and/or recovered from a component of its production environment, such that the “isolated” TCR is free or substantially free of other contaminant components from its production environment that might interfere with its therapeutic or diagnostic use. Contaminant components may include enzymes, hormones, and other proteinaceous or non-proteinaceous solutes. “Isolated” TCRs will thus be prepared by at least one purification step removing or substantially removing these contaminant components. The aforementioned definition is equally applicable to “isolated” polynucleotides/nucleic acids, mutatis mutandis.

### Soluble Forms

The TCRs of the present invention can be provided in soluble form. Soluble TCRs are useful as diagnostic tools, and carriers or “adapters” that specifically target therapeutic agents or effector cells to, for instance, a cancer cell expressing the antigenic target recognized by the soluble TCR. Soluble TCRs (sTCRs) will typically be fragments or constructs comprising TCR alpha and/or beta chains, or variable regions or CDRs thereof and optionally stabilized via disulfide bonds or covalently linked via a suitable linker molecule, e.g. as described above in the context of TCR constructs of the invention. They will typically not comprise e.g. a transmembrane region. In some circumstances amino acid modifications in the polypeptide sequence may be introduced in order to enhance solubility of the molecules, and/or correct folding and pairing of the alpha and beta chains (if desired), in particular when produced in a recombinant host that does not provide for the aforementioned features. For instance, when using E. coli as production host cells, folding and pairing of the TCR alpha and beta chains is typically accomplished in vitro. TCRs according to the invention may therefore for instance comprise additional cysteine residues, as described elsewhere herein.

Besides additional cysteine bridges, other useful modifications include, for instance, the addition of leucine zippers and/or ribosomal skipping sequences, e.g. sequence 2A from picorna virus as described in Walseng et al. (2015), PLoS ONE 10(4): e0119559 to increase folding, expression and/or pairing of the TCR alpha and/or beta chains.

### Modifications

The TCRs of the invention may further comprise one or more modifications as described in the following. The modifications described below will typically be covalent modifications and can be accomplished using standard techniques known in the art. In some circumstances, amino acid modifications in the TCRs may be required in order to facilitate the introduction of said modifications.

### Labels

The TCRs, in particular (soluble) TCRs, of the invention can be labelled. Useful labels are known in the art and can be coupled to the TCR or TCR variant using routine methods, optionally via linkers of various lengths. The term “label” or “labelling group” refers to any detectable label. In general, labels fall into a variety of classes, depending on the assay in which they are to be detected—the following examples include, but are not limited to: isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I); magnetic labels (e.g., magnetic particles); redox active moieties; optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluorophores or proteinaceous fluorophores; enzymatic groups (e.g. horseradish peroxidase, β-galactosidase, luciferase, alkaline phosphatase; biotinylated groups; or predetermined polypeptide epitopes recognized by a secondary reporter (e.g., leucine zipper pair sequences, binding sites for secondary antibodies, metal binding domains, epitope tags, etc.). Labelling is particularly envisaged when the TCRs, TCR variants or especially soluble TCR constructs (such as those comprising at least one TCR alpha and/or TCR beta chain as described herein) are intended for diagnostic use.

### Functional Moieties

The TCRs, in particular soluble TCRs, of the invention can be modified by attaching further functional moieties, e.g. for reducing immunogenicity, increasing hydrodynamic size (size in solution) solubility and/or stability (e.g. by enhanced protection to proteolytic degradation) and/or extending serum half-life.

Exemplary functional moieties for use in accordance with the invention include peptides or protein domains binding to other proteins in the human body (such as serum albumin, the immunoglobulin Fc region or the neonatal Fc receptor (FcRn), polypeptide chains of varying length (e.g., XTEN technology or PASylation®), non-proteinaceous polymers, including, but not limited to, various polyols such as polyethylene glycol (PEGylation), polypropylene glycol, polyoxyalkylenes, or copolymers of polyethylene glycol and polypropylene glycol, or of carbohydrates, such as hydroxyethyl starch (e.g., HESylation®) or polysialic acid (e.g., PolyXen® technology).

Other useful functional moieties include “suicide” or “safety switches” that can be used to shut off effector host cells carrying an inventive TCR in a patient's body. An example is the inducible Caspase 9 (iCasp9) “safety switch” described by Gargett and Brown Front Pharmacol. 2014; 5: 235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al, Proc Natl Acad Sci USA. 2008 Jan. 15; 105(2):623-8).

### Glycosylation

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Similarly, deglycosylation (i.e., removal of carbohydrate moieties present on the binding molecule) may be accomplished chemically, e.g. by exposing the TCRs to trifluoromethanesulfonic acid, or enzymatically by employing endo- and exo-glycosidases.

### Drug Conjugates

It is also conceivable to add a drug such as a small molecule compound to the TCRs, in particular soluble TCRs, of the invention. Linkage can be achieved via covalent bonds, or non-covalent interactions such as through electrostatic forces. Various linkers, known in the art, can be employed in order to form the drug conjugates.

### Tags

The TCRs, in particular soluble TCRs, of the invention can be modified to introduce additional domains which aid in identification, tracking, purification and/or isolation of the respective molecule (tags). Non-limiting examples of such tags comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strep II-tag), His-tag, CD20, Her2/neu tags, myc-tag, FLAG-tag, T7-tag, HA(hemagglutinin)-tag, or GFP-tags.

Epitope tags are useful examples of tags that can be incorporated into the TCR of the invention. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques. Examples of such techniques include: immunohistochemistry, immunoprecipitation, flow cytometry, immunofluorescence microscopy, ELISA, immunoblotting (“Western”), and affinity chromatography. The epitope tags can for instance have a length of 6 to 15 amino acids, in particular 9 to 11 amino acids. It is also possible to include more than one epitope tag in the TCR of the invention.

Tags can further be employed for stimulation and expansion of host cells carrying an inventive TCR by cultivating the cells in the presence of binding molecules (antibodies) specific for said tag. Nucleic acid

The present invention further provides nucleic acids encoding the TCRs described herein. Specifically, polynucleotides encoding TCR alpha or beta chains, TCR alpha or beta chain variable regions, and TCR CDR3alpha and CDR3beta, as well as TCR variants, constructs and fragments of the invention are provided herein.

The term “polynucleotide” or “nucleic acid” as used herein comprises a sequence of polyribonucleotides and polydeoxribonucleotides, e.g. modified or unmodified RNA or DNA, each in single-stranded and/or double-stranded form linear or circular, or mixtures thereof, including hybrid molecules. The nucleic acids according to this invention thus comprise DNA (such as dsDNA, ssDNA, cDNA), RNA (such as dsRNA, ssRNA, mRNA, ivtRNA), combinations thereof or derivatives (such as PNA) thereof.

A polynucleotide may comprise a conventional phosphodiester bond or a non-conventional bond (e.g., an amide bond, such as found in peptide nucleic acids (PNA)). The polynucleotides of the invention may also contain one or more modified bases, such as, for example, tritylated bases and unusual bases such as inosine. Other modifications, including chemical, enzymatic, or metabolic modifications, are also conceivable, as long as a binding molecule of the invention can be expressed from the polynucleotide. The polynucleotide may be provided in isolated form as defined elsewhere herein. A polynucleotide may include regulatory sequences such as transcription control elements (including promoters, enhancers, operators, repressors, and transcription termination signals), ribosome binding site, introns, or the like.

In particular, the present invention provides a polynucleotide comprising or consisting of a nucleic acid that is at least about 80%, about 85%, about 90%, about 91%, about 92%, about 93%, about 94%, about 95%, about 96%, about 97%, about 98%, about 99%, or 100% identical to a reference polynucleotide sequence selected from the group consisting of SEQ ID NOs: 23, 24, 25, 26, 27, 28, 29, or 30.

The polynucleotides described above may or may not comprise additional or altered nucleotide sequences encoding e.g., altered amino acid residues, a signal peptide to direct secretion of the encoded TCR, constant regions or other heterologous polypeptides as described herein. Such polynucleotides may thus encode fusion polypeptides, fragments, variants and other derivatives of the binding molecules described herein.

Also, the present invention includes compositions comprising one or more of the polynucleotides described above. Also provided herein are compositions, comprising a first polynucleotide and second polynucleotide wherein said first polynucleotide encodes a TCR alpha chain variable region as described herein and wherein said second polynucleotide encodes a TCR beta chain variable region as described herein.

The nucleic acid sequences of the present invention may be codon-optimized for optimal expression in the desired host cell, e.g. a human lymphocyte; or for expression in bacterial, yeast or insect cells that are particularly envisaged for the expression of soluble TCRs of the invention. Codon-optimization refers to the exchange in a sequence of interest of codons that are generally rare in highly expressed genes of a given species by codons that are generally frequent in highly expressed genes of such species, such codons encoding the same amino acids as the codons that are being exchanged. Selection of optimum codons thus depends on codon usage of the host genome and the presence of several desirable and undesirable sequence motifs.

### Vector

Further provided herein is a vector, comprising one or more of the polynucleotides as described herein. A “vector” is a nucleic acid molecule used as a vehicle to transfer (foreign) genetic material into a host cell where it can for instance be replicated and/or expressed.

The term “vector” encompasses, without limitation plasmids, viral vectors (including retroviral vectors, lentiviral vectors, adenoviral vectors, vaccinia virus vectors, polyoma virus vectors, and adenovirus-associated vectors (AAV)), phages, phagemids, cosmids and artificial chromosomes (including BACs and YACs). The vector itself is generally a nucleotide sequence, commonly a DNA sequence that comprises an insert (transgene) and a larger sequence that serves as the “backbone” of the vector. Engineered vectors typically comprise an origin for autonomous replication in the host cells (if stable expression of the polynucleotide is desired), selection markers, and restriction enzyme cleavage sites (e.g. a multiple cloning site, MCS). Vector may additionally comprise promoters, genetic markers, reporter genes, targeting sequences, and/or protein purification tags. As known to those skilled in the art, large numbers of suitable vectors are known to those of skill in the art and many are commercially available. Examples of suitable vectors are provided in J. Sambrook et al., Molecular Cloning: A Laboratory Manual (4th edition), Cold Spring Harbor Laboratory, Cold Spring Harbor Laboratory Press, New York (2012) and include

### Targeting Vectors

Targeting vectors can be used to integrate a polynucleotide into the host cell's chromosome by methods known in the art, such as described by J. Sambrook et al., Molecular Cloning: A Laboratory Manual (4th edition), Cold Spring Harbor Laboratory, Cold Spring Harbor Laboratory Press, New York (2012). Briefly, suitable means include homologous recombination or use of a hybrid recombinase that specifically targets sequences at the integration sites. Targeting vectors are typically circular and linearized before used for homologous recombination. As an alternative, the foreign polynucleotides may be DNA fragments joined by fusion PCR or synthetically constructed DNA fragments which are then recombined into the host cell. It is also possible to use heterologous recombination which results in random or non-targeted integration.

The present invention also provides a vector comprising the nucleic acid described herein.

### Expression Vectors

The vector of the present invention can also be an expression vector. “Expression vectors” or “expression constructs” can be used for the transcription of heterologous polynucleotide sequences, for instance those encoding the TCRs of the invention, and translation of their mRNA in a suitable host cell. This process is also referred to as “expression” of the TCRs of the invention herein.

Besides an origin of replication, selection markers, and restriction enzyme cleavage sites, expression vectors typically include one or more regulatory sequences operably linked to the heterologous polynucleotide to be expressed.

The term “regulatory sequence” refers to a nucleic acid sequence necessary for the expression of an operably linked coding sequence of a (heterologous) polynucleotide in a particular host organism or host cell and thus include transcriptional and translational regulatory sequences. Typically, regulatory sequences required for expression of heterologous polynucleotide sequences in prokaryotes include a promoter(s), optionally operator sequence(s), and ribosome binding site(s). In eukaryotes, promoters, polyadenylation signals, enhancers and optionally splice signals are typically required. Moreover, specific initiation and secretory signals also may be introduced into the vector in order to allow for secretion of the polypeptide of interest into the culture medium.

A nucleic acid is “operably linked” when it is placed into a functional relationship with another nucleic acid sequence, in particular on the same polynucleotide molecule. For example, a promoter is operably linked with a coding sequence of a heterologous gene when it is capable of effecting the expression of that coding sequence. The promoter is typically placed upstream of the gene encoding the polypeptide of interest and regulates the expression of said gene.

Exemplary regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. As set out before, the expression vectors may also include origins of replication and selectable markers.

As mentioned previously, vectors of the invention may further comprise one or more selection markers. Suitable selection markers for use with eukaryotic host cells include, without limitation, the herpes simplex virus thymidine kinase (tk), hypoxanthine-guanine phosphoribosyltransferase (hgprt), and adenine phosphoribosyltransferase (aprt) genes. Other genes include dhfr (methotrexate resistance), gpt (mycophenolic acid resistance) neo (G-418 resistance) and hygro (hygromycin resistance). Vector amplification can be used to increase expression levels. In general, the selection marker gene can either be directly linked to the polynucleotide sequences to be expressed, or introduced into the same host cell by co-transformation.

In view of the above, the present invention thus further provides one or more of the nucleotide sequences described herein inserted into (i.e. comprised by) a vector. Specifically, the invention provides (replicable) vectors comprising a nucleotide sequence encoding a TCR of the invention, or an alpha or beta chain thereof, or an alpha or beta variable domain, or a CDR3 alpha or CDR3beta operably linked to a promoter.

The skilled person will readily be able to select a suitable expression vector based on, e.g., the host cell intended for TCR expression. Examples for suitable expression vectors are viral vectors, such as retroviral vectors e.g. MP71 vectors or retroviral SIN vectors; and lentiviral vectors or lentiviral SIN vectors. Viral vectors comprising polynucleotides encoding the TCRs of the invention are for instance capable of infecting lymphocytes, which are envisaged to subsequently express the heterologous TCR. Another example for a suitable expression vector is the Sleeping Beauty (SB) transposon transposase DNA plasmid system, SB DNA plasmid. The nucleic acids and/or in particular expression constructs of the invention can also be transferred into cells by transient RNA transfection.

Currently used viral vectors for native TCR expression typically link the TCR-alpha and TCR-beta chain genes in one vector with either an internal ribosomal entry site (IRES) sequence or the 2A peptide sequence derived from a porcine tsechovirus, resulting in the expression a single messenger RNA (mRNA) molecule under the control of the viral promoter within the transduced cell.

### Host Cell

The present invention further provides a host cell comprising the TCR, nucleic acid or the vector described herein.

A variety of host cells can be used in accordance with the invention. As used herein, the term “host cell” encompasses cells which can be or has/have been recipients of polynucleotides or vectors described herein and/or express (and optionally secreting) the TCR of the present invention. The terms “cell” and “cell culture” are used interchangeably to denote the source of a TCR unless it is clearly specified otherwise. The term “host cell” also includes “host cell lines”.

In general, the term “host cell” includes prokaryotic or eukaryotic cells, and also includes without limitation bacteria, yeast cells, fungi cells, plant cells, and animal cells such as insect cells and mammalian cells, e.g., murine, rat, macaque or human cells.

In view of the above, the invention thus provides, inter alia, host cells comprising a polynucleotide or a vector, e.g. an expression vector comprising a nucleotide sequence encoding a TCR or TCR construct as described herein.

Polynucleotides and/or vectors of the invention can be introduced into the host cells using routine methods known in the art, e.g. by transfection, transformation, or the like.

“Transfection” is the process of deliberately introducing nucleic acid molecules or polynucleotides (including vectors) into target cells. An example is RNA transfection, i.e. the process of introducing RNA (such as in vitro transcribed RNA, ivtRNA) into a host cell. The term is mostly used for non-viral methods in eukaryotic cells. The term “transduction” is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside. Exemplary techniques for transfecting eukaryotic host cells include lipid vesicle mediated uptake, heat shock mediated uptake, calcium phosphate mediated transfection (calcium phosphate/DNA co-precipitation), microinjection and electroporation.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylene glycol mediated uptake.

In view of the above, the present invention thus further provides host cells comprising at least one polynucleotide sequence and/or vector as described herein.

For expression of the TCRs of the invention, a host cell may be chosen that modulates the expression of the inserted polynucleotide sequences, and/or modifies and processes the gene product (i.e. RNA and/or protein) as desired. Such modifications (e.g., glycosylation) and processing (e.g., cleavage) of gene products may be important for the function of the TCR. Different host cells have characteristic and specific mechanisms for the post-translational processing and modification of gene products. Appropriate cell lines or host systems can be chosen to ensure the correct modification and processing of the product. To this end, eukaryotic host cells that possess the cellular machinery for proper processing of the primary transcript, glycosylation, and phosphorylation of the gene product may be used.

It is envisaged herein to provide (a) host cells for expressing and obtaining TCRs of the invention, in particular in soluble form (“production host cells”) and (b) host cells expressing a TCR of the invention and having effector function (“effector host cells”). Such “effector host cells” are particularly useful for therapeutic applications and are envisaged for administration to a subject in need thereof. Preferred “effector host cells” include lymphocytes such as cytotoxic T lymphocytes (CTLs), CD8+ T cells, CD4+ T cells, natural killer (NK) cells, natural killer T (NKT) cells, gamma/delta-T-cells.

### “Production Host Cell”

### Cells

“Production host cells” used for the expression of soluble TCRs of the invention are preferably capable of expressing high amounts of recombinant protein.

Exemplary mammalian host cells that can be used for as “production host cells” include Chinese Hamster Ovary (CHO cells) including DHFR minus CHO cells such as DG44 and DUXBI 1, NSO, COS (a derivative of CVI with SV40 T antigen), HEK293 (human kidney), and SP2 (mouse myeloma) cells. Other exemplary host cell lines include, but are not limited to, HELA (human cervical carcinoma), CVI (monkey kidney line), VERY, BHK (baby hamster kidney), MDCK, 293, W138, R1610 (Chinese hamster fibroblast) BALBC/3T3 (mouse fibroblast), HAK (hamster kidney line), P3x63-Ag3.653 (mouse myeloma), BFA-IcIBPT (bovine endothelial cells), and RAJI (human lymphocyte). Host cell lines are typically available from commercial services, the American Tissue Culture Collection (ATCC) or from published literature.

Non-mammalian cells such as bacterial, yeast, insect or plant cells are also readily available and can also be used as “production host cells” as described above. Exemplary bacterial host cells include enterobacteriaceae, such Escherichia coli, Salmonella; Bacillaceae, such as Bacillus subtilis; Pneumococcus; Streptococcus, and Haemophilus influenza. Other host cells include yeast cells, such as Saccharomyces cerevisiae, and Pichia pastoris. Insect cells include, without limitation, Spodoptera frugiperda cells.

In accordance with the foregoing, conceivable expressions systems (i.e. host cells comprising an expression vector as described above) include microorganisms such as bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid). Mammalian expression systems harboring recombinant expression constructs containing promoters derived from the genome of mammalian cells (e.g., metallothionein promoter) or from mammalian viruses (e.g., the adenovirus late promoter; the vaccinia virus 7.5K promoter, the cytomegalovirus (CMV) major immediate-early promoter (MIEP) promoter) are often preferred. Suitable mammalian host cells can be selected from known cell lines (e.g., COS, CHO, BLK, 293, 3T3 cells), however it is also conceivable to use lymphocytes such as cytotoxic T lymphocytes (CTLs), CD8+ T cells, CD4+ T cells, natural killer (NK) cells, natural killer T (NKT) cells, gamma/delta-T-cells.

In accordance with the foregoing, the present invention also provides a method for producing and obtaining a TCR as described herein comprising the steps of (i) incubating a host cell (i.e., a production host cell) under conditions causing expression of said TCR and (ii) purifying said TCR.

### Cultivation

The host cells harboring the expression vector are grown under conditions appropriate to the production of the TCRs provided herein, in particular alpha chains and/or beta chains as described elsewhere herein, and assayed for alpha and/or beta chain protein synthesis. For the expression of double-chained TCRs, vectors encoding both the alpha and beta chains may be co-expressed in the host cell for expression of the entire molecule.

### Purification

Once a TCR of the invention has been expressed, it may be purified by any purification method known in the art, for example, by chromatography (e.g., ion exchange chromatography (e.g. hydroxylapatite chromatography), affinity chromatography, particularly Protein A, Protein G or lectin affinity chromatography, sizing column chromatography), centrifugation, differential solubility, hydrophobic interaction chromatography, or by any other standard technique for the purification of proteins. The skilled person will readily be able to select a suitable purification method based on the individual characteristics of the TCR to be recovered.

### “Effector Host Cell”

As mentioned earlier, the present invention also provides for “effector host cells” comprising a nucleotide sequence, vector or TCR of the invention. Said effector host cells are modified using routine methods to comprise a nucleic acid sequence encoding the TCR of the invention, and are envisaged to express the TCR described herein, in particular on the cell surface. For the purposes of the present invention, “modified host cells expressing a TCR of the invention” generally refers to (effector or production) host cells treated or altered to express a TCR according to the present invention, for instance by RNA transfection as described in the appended Examples. Other methods of modification or transfection or transduction, such as those described elsewhere herein, are also envisaged. The term “modified host cell” thus includes “transfected”, “transduced” and “genetically engineered” host cells preferably expressing the TCR of the present invention.

Preferably, such “(modified) effector host cells” (in particular “(modified) effector lymphocytes”) are capable of mediating effector functions through intracellular signal transduction upon binding of the TCR to its specific antigenic target. Such effector functions include for instance the release of perforin (which creates holes in the target cell membrane), granzymes (which are proteases that act intracellularly to trigger apoptosis), the expression of Fas ligand (which activates apoptosis in a Fas-bearing target cell) and the release of cytokines, preferably Th1/Tc1 cytokines such as IFN-γ, IL-2 and TNF-α. Thus, an effector host cell engineered to express the TCR of the invention that is capable recognizing and binding to its antigenic target in the subject to be treated is envisaged to carry out the above-mentioned effector functions, thereby killing the target (e.g. cancer) cells. Cytolysis of target cells can be assessed e.g. with the CTL fluorescent killing assay (CTL, USA) detecting the disappearance of fluorescently labeled target cells during co-culture with TCR-transfected recipient T cells.

In view of the above, effector host cells preferably express a functional TCR, i.e. that typically comprises a TCR alpha and beta chain described herein; and also the signal transducing subunits CD3 gamma, delta, epsilon and zeta (CD3 complex). Moreover, expression of co-receptors CD4 or CD8 may also be desired. Generally, lymphocytes harboring the required genes involved in antigen binding, receptor activation and downstream signalling (e.g. Lck, FYN, CD45, and/or Zap70), T cells are particularly suitable as effector host cells. However, effector host cells expressing the TCR of the invention as a “binding domain” without the CD3 signal transducing subunit and/or aforementioned downstream signalling molecules (i.e. being capable of recognizing the antigenic target described herein, but without effecting functions mediated by CD3 and/or the aforementioned downstream signalling molecules) are also envisaged herein. Such effector cells are envisaged to be capable of recognizing the antigenic target described herein, and optionally of effecting other functions not associated with CD3 signalling and/or signalling of the aforementioned downstream signalling molecules. Examples include NK or NKT cells expressing the inventive TCR and being capable of e.g. releasing cytotoxic granules upon recognition of their antigenic target.

Thus, cytotoxic T lymphocytes (CTLs), CD8+ T cells, CD4+ T cells, natural killer (NK) cells, natural killer T (NKT) cells, gamma/delta-T-cells are considered useful lymphocyte effector host cells. Such lymphocytes expressing the recombinant TCR of the invention are also referred to as “modified effector lymphocytes” herein. The skilled person will however readily acknowledge that in general any component of the TCR signalling pathway leading to the desired effector function can be introduced into a suitable host cell by recombinant genetic engineering methods known in the art.

Effector host cells in particular lymphocytes such as T cells can be autologous host cells that are obtained from the subject to be treated and transformed or transduced to express the TCR of the invention. Typically, recombinant expression of the TCR will be accomplished by using a viral vector as described in the appended Examples. Techniques for obtaining and isolating the cells from the patient are known in the art.

As mentioned earlier, the effector host cells provided herein are particularly envisaged for therapeutic applications. Further genetic modifications of the host cells may be desirable in order to increase therapeutic efficacy. E.g., when using autologous CD8+ T cells as “effector host cells” suitable additional modifications include downregulation of the endogenous TCR, CTLA-4 and/or PD-1 expression; and/or amplification of co-stimulatory molecules such as CD28, CD134, CD137. Means and methods for achieving the aforementioned genetic modifications have been described in the art.

Methods for targeted genome engineering of host cells are known in the art and include, besides gene knockdown with siRNA, the use of so-called “programmable nucleases” such as zinc-finger nucleases (ZFNs), transcription activator-like effector nucleases (TALENs) and RNA-guided engineered nucleases (RGENs) derived from the bacterial clustered regularly interspaced short palindromic repeat (CRISPR)-Cas (CRISPR-associated) system, as inter alia reviewed in Kim & Kim Nature Reviews Genetics 15, 321-334 (2014). For instance, programmable nucleases such as TALENs can be employed to cut the DNA regions that code for “unwanted” proteins, such as PD-1, CTLA-4 or an endogenous TCR, and thereby reducing their expression. When T cells are used as (effector) host cells, downregulation of the endogenous TCR has the benefit of reducing unwanted “mispairing” of endogenous and exogenous TCR alpha/beta chains.

### Pharmaceutical Composition/Diagnostic

The present invention further provides a pharmaceutical composition as one or more active agents, the TCR, the nucleic acid, the vector and/or the host cell as described herein, and, optionally, one or more pharmaceutically excipient(s). Accordingly, the use of said TCR, nucleic acid, vector and host cell for the manufacture of a pharmaceutical composition or medicament is also envisaged herein.

The term “pharmaceutical composition” particularly refers to a composition suitable for administering to a human. However, compositions suitable for administration to non-human animals are generally also encompassed by the term.

The pharmaceutical composition and its components (i.e. active agents and optionally excipients) are preferably pharmaceutically acceptable, i.e. capable of eliciting the desired therapeutic effect without causing any undesirable local or systemic effects in the recipient. Pharmaceutically acceptable compositions of the invention may for instance be sterile. Specifically, the term “pharmaceutically acceptable” may mean approved by a regulatory agency or other generally recognized pharmacopoeia for use in animals, and more particularly in humans.

The active agent described in the foregoing (for instance the host cell or the TCR) is preferably present in the pharmaceutical composition in a therapeutically effective amount. By “therapeutically effective amount” is meant an amount of the active agent that elicits the desired therapeutic effect. Therapeutic efficacy and toxicity can be determined by standard procedures, e.g. in cell culture or in test animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred.

### Dosage

The exact dosage of the TCR polynucleotide, vector or host cell will be ascertainable by one skilled in the art using known techniques. Suitable dosages provide sufficient amounts of the active agent of the invention and are preferably therapeutically effective, i.e. elicit the desired therapeutic effect.

As is known in the art, adjustments for purpose of the treatment (e.g. remission maintenance vs. acute flare of disease), route, time and frequency of administration, time and frequency of administration formulation, age, body weight, general health, sex, diet, severity of the disease state, drug combination(s), reaction sensitivities, and tolerance/response to therapy may be necessary. Suitable dosage ranges, for instance for soluble TCRs as described herein, can be determined using data obtained from cell culture assays and animal studies and may include the ED₅₀. Typically, dosage amounts may vary from 0.1 to 100000 micrograms, up to a total dose of about 2 g, depending upon the route of administration. Exemplary dosages of the active agent of the invention are in the range from about 0.01 mg/kg to about 10 mg/kg, from about 0.1 mg/kg to about 10 mg/kg, from about 1 mg/kg to about 10 mg/kg, from about 1 mg/kg to about 5 mg/kg, from about 0.01 mg/kg to about 1 mg/kg, or from about 0.1 mg/kg to about 1 mg/kg. Guidance as to particular dosages and methods of delivery is provided in the literature. It is recognized that treatment may require a single administration of a therapeutically effective dose or multiple administrations of a therapeutically effective dose of the active agent of the invention. E.g., some pharmaceutical compositions might be administered every 3 to 4 days, every week, or once every two weeks, or once within a month depending on formulation, half-life and clearance rate of the particular composition.

As set out previously, the pharmaceutical composition may optionally comprise one or more excipients and/or additional active agents.

### Excipients

The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. It is within the knowledge of the skilled person to select suitable excipients for preparing the desired pharmaceutical composition of the invention. Exemplary carriers for use in the pharmaceutical composition of the invention include saline, buffered saline, dextrose, and water. Typically, choice of suitable excipients will inter alia depend on the active agent used, the disease to be treated, and the desired formulation of the pharmaceutical composition.

### Additional Active Agents

The present invention further provides pharmaceutical compositions comprising one or more of the inventive active agents specified above (for instance a host cell or a TCR construct), and one or more additional active agents that are suitable for treatment and/or prophylaxis of the disease to be treated. Preferred examples of active ingredients suitable for combinations include known anti-cancer drugs such as cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolmide, topotecan, trimetreate glucuronate, auristatin E vincristine and doxorubicin; and peptide cytotoxins such as ricin, diphtheria toxin, pseudomonas bacterial exotoxin A, DNAase and RNAase; radio-nuclides such as iodine 131, rhenium 186, indium 111, yttrium 90, bismuth 210 and 213, actinium 225 and astatine 213; prodrugs, such as antibody directed enzyme pro-drugs; immuno-stimulants, such as IL-2, chemokines such as IL-8, platelet factor 4, melanoma growth stimulatory protein, etc., antibodies or fragments thereof such as anti-CD3 antibodies or fragments thereof, complement activators, xenogeneic protein domains, allogeneic protein domains, viral/bacterial protein domains and viral/bacterial peptides.

### Administration

A variety of routes are applicable for administration of the pharmaceutical composition according to the present invention. Typically, administration will be accomplished parentally. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration.

### Formulation

The pharmaceutical compositions of the invention can be formulated in various forms, depending inter alia on the active agent used (e.g., soluble TCR), e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. Processes known per se for producing medicaments are indicated in 22nd edition of Remington's Pharmaceutical Sciences (Ed. Maack Publishing Co, Easton, Pa., 2012) and may include, for instance conventional mixing, dissolving, granulating, dragee-making, levigating, emulsifying, encapsulating, entrapping or lyophilizing processes. Pharmaceutical compositions comprising, for instance, host cells or soluble TCR as described herein will typically be provided in a liquid form, and preferably comprise a pharmaceutically acceptable buffer.

After pharmaceutical compositions of the invention have been prepared they can be placed in an appropriate container and labeled for treatment of an indicated condition. Such labeling would for instance include amount, frequency and method of administration.

### Treatment

In view of the foregoing the present invention thus provides a TCR, nucleic acid, vector and/or host cell as described herein for use as a medicament.

The TCR, nucleic acid, vector and/or host cell can in general be employed for treatment detection, diagnosis, prognosis, prevention and/or treatment of diseases or disorders. The term “treatment” in all its grammatical forms includes therapeutic or prophylactic treatment of a subject in need thereof. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

The terms “subject” or “individual” or “animal” or “patient” are used interchangeably herein to refer to any subject, particularly a mammalian subject, for whom therapy is desired. Mammalian subjects generally include humans, non-human primates, dogs, cats, guinea pigs, rabbits, rats, mice, horses, cattle, cows, and the like. However, it will readily be understood that the TCRs, nucleic acids, vectors, host cells and pharmaceutical compositions provided herein are especially envisaged for treatment of human subjects, in particular those that are HLA-A2-positive.

### Direct Administration

For therapy, TCRs—in particular soluble TCRs of the invention—, nucleic acids, vectors (such as viral vectors) or host cells of the invention can be administered directly to the subject in need thereof. Thus the present invention provides a TCRs, nucleic acid, vector or host cells for use in a method of detecting, diagnosing, prognosing, preventing and/or treating of cancer. Said method can comprise the steps of (a) providing one or more of (i) a TCR (ii), a nucleic acid, (iii) a vector, (iv) a host cell, and/or (v) a pharmaceutical composition of the present invention; and (b) administering one or more of (i)-(v) to the subject in need thereof. Optionally, the method can comprise a further step of cancer therapy, e.g. radiation, or administration of one or more anti-cancer agents.

### Ex Vivo Treatment

Treatment according to the invention may also comprise the steps of (a) providing a sample of a subject, said sample comprising lymphocytes; (b) providing one or more of the TCR, nucleic acid, vector host cell and/or pharmaceutical composition of the invention (c) introducing of one or more of (i) to (v) of step (b) into the lymphocytes of step (a) and, thereby, obtaining modified lymphocytes, (d) administering the modified lymphocytes of step (c) to a subject or patient in need thereof.

The lymphocytes provided in step (a) are particularly envisaged to be “effector host cells” as described in the foregoing and are advantageously selected from T cells, NK cells and/or NKT cells, especially CD8+ T cells; and can be obtained in a previous step (a′) from a sample—in particular a blood sample of the subject by routine methods known in the art. It is however also conceivable to use other lymphocytes that are preferably capable of expressing the TCR of the present invention and exert the desired biological effector functions as described herein. Moreover, said lymphocytes will typically be selected for compatibility with the subject's immune system, i.e. they will preferably not elicit an immunogenic response. For instance, it is conceivable to use a “Universal Recipient Cells”, i.e. universally compatible lymphocytes exerting the desired biological effector functions that can be grown and expanded in vitro. Use of such cells will thus obviate the need for obtaining and providing the subject's own lymphocytes in step (a).

The ex vivo introduction of step (c) can be carried out by introducing a nucleic acid or vector described herein via electroporation into the lymphocytes, or by infecting the lymphocytes with a viral vector, such as a lentiviral or retroviral vector as described previously in the context of the effector host cell. Other conceivable methods include the use of by transfection reagents, such as liposomes, or transient RNA transfection. The transfer of antigen-specific TCR genes into (primary) T cells by e.g. (retro-)viral vectors or transient RNA transfection represents a promising tool for generating tumor-associated antigen-specific T cells that can subsequently be re-introduced into the donor, where they specifically target and destroy tumor cells expressing said antigen. In the present invention, said tumor-associated antigen is PRAME₁₀₀₋₁₀₈, particularly in its HLA-A*02 bound form.

In view of the above, a further aspect of the present invention is thus the use of a TCR, a nucleic acid sequence, a vector and/or a host cell as described elsewhere herein for generating modified lymphocytes. Means and methods for introducing, e.g. a nucleic acid and a vector into the lymphocytes have been described elsewhere herein.

### Diagnostic Composition

The present invention also provides a diagnostic composition comprising, as one or more diagnostic agent(s), the TCR, nucleic acid, the vector and/or the host cell as described herein. Typically, said diagnostic agent will comprise means for detecting its binding to its antigenic target, for instance a label as described in the context of the TCR constructs of the invention. As regards the host cell, it is for instance conceivable to use modified host cells comprising a dye or a contrast agent that is released (instead of cytotoxic granules) upon antigen recognition.

### Use

The present invention envisages use of the diagnostic agents described in the foregoing for detecting, diagnosing and/or prognosing cancer in a subject which can be accomplished in vivo or in vitro.

Thus the invention provides a diagnostic composition for use in detecting, diagnosing and/or pronging cancer in a subject in vivo, said composition comprising, as a diagnostic agent, the TCR, the nucleic acid, the vector and/or the host cell of the invention. The method will typically comprise (a) administering said diagnostic agent to the subject and (b) detecting binding of said diagnostic agent to its antigenic target.

Moreover, the invention provides a method of detecting, diagnosing and/or prognosing cancer in a subject in vitro. In accordance the present invention also provides a method of detecting the presence of a cancer in a subject, comprising the steps of (a) providing a sample of a subject, said sample comprising one or more cells; (b) contacting said sample with the TCR, nucleic acid, vector and/or host cell of the invention; thereby forming a complex, and (c) detecting the complex. Said complex is envisaged to be indicative for binding of the diagnostic agent to its antigenic target and is of the presence of a (cancer) cell expressing said antigenic target.

In both methods binding of the diagnostic agent to its antigenic target is detectable by using routine methods known in the art and will inter alia depend on the specific diagnostic agent used. Suitable labels that can be coupled to the diagnostic agent of the invention are exemplified in the section relating to labeled TCR constructs. Use for generating modified lymphocytes.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., “about 20” includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following example, offered for illustrative purposes only. The example is not intended to limit the scope of the present invention in any way.

The invention is also characterized by the following items:
1. A T-cell receptor (TCR) comprising:
(i) a CDR3 of the TCR alpha chain variable region comprising or consisting of the amino acid sequence of CAVHSTAQAGTALIF (SEQ ID NO: 1)
or an amino acid sequence having at least 80% identity to SEQ ID NO 1, more preferably at least 85% identity, more preferably 90% or 95% and/or
(ii) a CDR3 of the TCR beta chain variable region comprising or consisting of the amino acid sequence of CASSTHRGQTNYGYTF (SEQ ID NO. 2)
or an amino acid sequence having at least 80% identity to SEQ ID NO: 2, more preferably at least 85% identity, more preferably 90% or 95% identity.
2. The TCR according to item 1, said TCR being capable of binding to the epitope comprised within the amino acid sequence of X1 LX2GLDX3LL (SEQ ID NO:31) or its MHC-bound form, preferably to the epitope comprised within the amino acid sequence of VLDGLDVLL (SEQ ID NO:32) or its MHC-bound form.
3. The TCR according to any one of items 1 or 2, comprising
(i) a TCR alpha chain variable region comprising or consisting of the amino acid sequence depicted in SEQ ID NO: 15, and/or
(ii) a TCR beta chain variable region comprising or consisting of the amino acid sequence depicted in SEQ ID NO: 16.
4. The TCR according to any one of the preceding items, further comprising
(i) a TCR alpha chain constant region and/or
(ii) a TCR beta chain constant region
5. The TCR according to any one of the preceding items, comprising
(i) a TCR alpha chain comprising or consisting of an amino acid sequence selected from SEQ ID NO: 7; SEQ ID NO: 9, SEQ ID NO: 11, or SEQ ID NO: 13,
or an amino acid sequence having at least 80% identity, more preferably at least 85% identity, more preferably 90% or 95% to SEQ ID NO: 7, 11, 9 or 13; and/or
(ii) a TCR beta-chain comprising or consisting of an amino acid sequence selected from of SEQ ID NO: 8, SEQ ID NO: 10, SEQ ID NO: 12, or SEQ ID NO: 14
or an amino acid sequence having at least 80% identity, more preferably at least 85% identity, more preferably 90% or 95% to SEQ ID NO: 8, 10, 12 or 14.
6. The TCR according to any one of the preceding items, said TCR being selected from a native TCR, a TCR variant, a TCR fragment, or a TCR construct.
7. The TCR construct according to item 6, comprising at least one TCR alpha-chain(s) and at least one TCR beta-chain(s) covalently linked to each other to form TCR heterodimers or multimers.
8. The TCR according to any of the preceding items, further comprising one or more fusion component(s) optionally selected from Fc receptors; Fc domains, including IgA, IgD, IgG, IgE, and IgM; cytokines, including IL-2 or IL-15; toxins; antibodies or antigen-binding fragments thereof, including anti-CD3, anti-CD28, anti-CD5, anti-CD 16 or anti-CD56 antibodies or antigen-binding fragments thereof; CD247 (CD3-zeta), CD28, CD137, CD134 domain, or combinations thereof, optionally further comprising at least one linker.
9. The TCR according to any one of the preceding items, comprising
   (i) at least one TCR alpha-chain as defined in any one of items 1 to 4; and
   (ii) at least one TCR beta-chain as defined in any one of items 1 to 4
   (iii) an antibody or a single chain antibody fragment (scFv) which is directed against an antigen or epitope on the surface of lymphocytes.
wherein the TCR alpha-chain(s) and TCR beta-chain(s) are linked to each other and fused, optionally via a linker, to said antibody or scFv.
10. The TCR according to item 9, wherein said antigen is selected from CD3, CD28, CD5, CD16 or CD56.
11. The TCR according to any one of the preceding items, further comprising at least one label.
12. The TCR according to any one of the preceding items which is soluble.
13. A nucleic acid encoding the TCR according to any one of the preceding items.
14. The nucleic acid according to item 13, comprising the nucleic acid sequence of SEQ ID NO: 23, 24, 25, 26, 27, 28, 29 or 30.
15. A vector comprising the nucleic acid according to any one of items 13 or 14.
16. A host cell comprising the TCR according to any one of items 1 to 12, the nucleic acid sequence according to item 12 or 13 or the vector according to item 14.
17. The host cell according to item 16 which is selected from lymphocytes including but not limited to cytotoxic T lymphocytes (CTLs), CD8+ T cells, CD4+ T cells, natural killer (NK) cells, natural killer T (NKT) cells, gamma/delta-T-cells.
18. A method for obtaining a TCR according to any one of items 1 to 12, comprising (i) incubating a host cell according to item 17 under conditions causing expression of said TCR (ii) purifying said TCR.
19. A pharmaceutical or diagnostic composition comprising one or more of:
   (i) the TCR according to any one of items 1 to 12;
   (ii) the nucleic acid according to any one of items 13 to 14
   (iii) the vector according to item 15; and/or
   (iv) the host cell according to any one of items 16 or 17,
and, optionally, pharmaceutically excipient(s).
20. The TCR according to any one of items 1 to 12, the nucleic acid according to item 13 or 14, the vector according to item 15 and/or the host cell according to item 16 or 17 for use as a medicament.
21. The TCR according to any one of items 1 to 12, the nucleic acid according to item 13 or 14, the vector according to item 15 and/or the host cell according to item 16 or 17 for use as a medicament. for use in detection, diagnosis, prognosis, prevention and/or treatment of cancer.
22. The TCR, nucleic acid, vector and/or host cell for the use of item 21, wherein prevention and/or treatment of cancer comprises
(a) providing or more of
   (i) the TCR according to any one of items 1 to 12;
   (ii) the nucleic acid according to any one of items 13 or 14
   (iii) the vector according to item 15; and/or
   (iv) the host cell according to any one of items 16 or 17,
   (v) the pharmaceutical composition according to item 19; and
(b) administering at least one of (i) to (v) to a subject in need thereof.
23. The TCR, nucleic acid, vector and/or host cell for the use of item 21, wherein prevention and/or treatment of cancer comprises
(a) providing a sample of a subject, said sample comprising lymphocytes;
(b) providing one or more of
   (i) the TCR according to any one of items 1 to 12;
   (ii) the nucleic acid according to any one of items 13 or 14
   (iii) the vector according to item 15; and/or
   (iv) the host cell according to any one of items 16 or 17,
   (v) the pharmaceutical composition according to item 19;
(c) introducing of one or more of (i) to (v) of step (b) into the lymphocytes of step (a) and, thereby, obtaining modified lymphocytes,
(d) administering the modified lymphocytes of step (c) to a subject or patient in need thereof.
24. A method of detecting the presence of a cancer in a subject in vitro, comprising:
(a) providing a sample of a subject, said sample comprising one or more cells;
(b) contacting said sample with
   (i) the TCR according to any one of items 1 to 12;
   (ii) the nucleic acid according to any one of items 13 or 14
   (iii) the vector according to item 15; and/or
   (iv) the host cell according to any one of items 16 or 17,
   (v) the pharmaceutical composition according to item 19;
thereby forming a complex, and
(c) detecting the complex,
wherein detection of the complex is indicative of the presence of the cancer in the subject.
25. Use of a TCR according to any one of items 1 to 12, a nucleic acid according to item 13 or 14 and/or a vector according to item 15 for generating modified lymphocytes.

### EXAMPLES

### Abbreviations and Synonyms

(m)DC (mature) dendritic cell
ivtRNA In vitro transcribed RNA
APC antigen-presenting cell
(X)^{pos} or (X)⁺ expressing X
VLD or VLD peptide PRAME₁₀₀₋₁₀₈
E:T ratio Ratio of effector cells to target cells
SLL or SLL peptide Peptide, irrelevant, SLLQHLIGL (SEQ ID NO: 229)
ALY or ALY peptide Peptide, irrelevant, ALYVDSLFFL (SEQ ID NO: 230)
ELA or ELA peptide Peptide, irrelevant, ELAGIGILTV (SEQ ID NO: 231)
[M] Concentration molar [mol/L]
PBMC Peripheral blood mononuclear cell, i.e. nucleated cells in the peripheral blood; comprise PBL (peripheral blood lymphocytes) such as T cells.

### Example 1: Isolation of PRAME-Specific T Cell Clone

The present inventors used an in vitro priming approach to isolate T cell clones of any desired MHC restriction and antigen specificity. The priming system uses mature dendritic cells (mDCs) as antigen-presenting cells and autologous CD8⁺-enriched T cells as responding cells. In vitro transcribed RNA (ivtRNA) encoding the full-length human PRAME amino acid sequence as referenced in SEQ ID NO: 33 serves as the source of specific antigen. After electroporation into the mDCs the ivtRNA is translated into full-length protein, which is subsequently processed and presented as peptides by the MHC molecules of the mDCs. In vitro co-cultures of T cells with the ivtRNA-transfected mDCs from the same donor leads to de novo induction of antigen-specific T cells that serve as the source of corresponding TCRs. Antigen-specific T cells can be enriched by a variety of methods and are cloned by limiting dilution or FACS-based single cell plating.

### Example 1.1: Priming Approach Using Mature Dendritic Cells

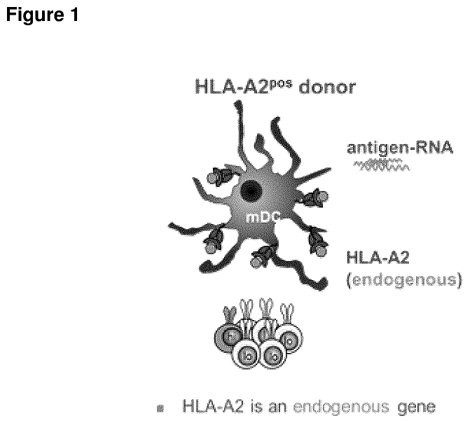

DC priming of T cells with high-affinity TCR was accomplished using peptide presentation by autologous MHC molecules according to the following protocol (FIG. 1):
HLA-A*02:01/PRAME priming
8 days mDCs produced using suitable maturation cocktails for DCs
APC loading: ivtRNA
Enrichment via HLA-A*02:01 PRAME₁₀₀₋₁₀₈ multimer
Single cell sorting using FACS technology

### Example 2: Function/Specificity Analyses

Following identification of a candidate T cell clone (T cell clone T4.8-1-29) that recognizes the desired PRAME epitope (PRAME₁₀₀₋₁₀₈), full characterization regarding function and specificity was conducted. Analyses included the cytokine secretion pattern of the isolated T cell clone (T cell clone T4.8-1-29) in co-culture with various human tumor cell lines, the capacity of the clone to specifically recognize various target cells, the functional avidity of the clone and cytotoxicity towards T2 and tumor cells.

### Example 2.1: Analysis of the Original T Cell Clone T4.8-1-29

Example 2.1.1: Poly-Cytokine Analysis
   Experimental Layout: Stimulation by Peptide-Loaded T2 Cells Cytokine release was measured according to the following protocol:

Multiplex® cytokine analysis was performed, detecting IFN-gamma, IL-2, TNF-alpha, IL-5, IL-10, IL-6, IL12p70, IL-4 and IL-1beta

Stimulating cells: T2 cells (HLA-A*02^{pos}) loaded with saturating amounts (10⁻⁵ M) of PRAME₁₀₀₋₁₀₈ peptide (“VLD peptide”) or irrelevant PRAME₃₀₀₋₃₀₉, i.e. ALYVDSLFFL peptide (“ALY peptide”, SEQ ID NO: 230)

Supernatants of T cell co-cultures, with relevant or irrelevant peptide-loaded T2 cells, were harvested after 24 h and subsequently measured using Multiplex® cytokine analysis.

### Results

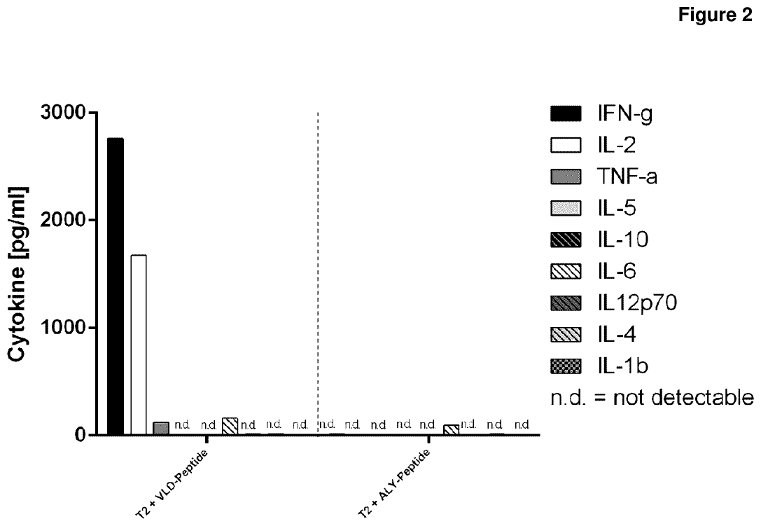

The candidate clone secreted IFN-gamma, IL-2 and TNF-alpha (Th1/Tc1 cytokines) above background levels. The cytokine expression pattern reflects a Th1 phenotype that is related to good anti-tumor effector function (FIG. 2).

IL-5 and IL-13 (Th2/Tc2 cytokines) secretion was not detected (n.d.).

### Example 2.2: Recognition of Tumor Cells

### Experimental Layout: Stimulation by Tumor Cell Lines

IFN-gamma ELISA was used to assess cytokine secretion after stimulation with a panel of human tumor cell lines (status of PRAME expression was detected by NanoString® nCounter® (Nanostring Technologies, Inc.) analysis).

Supernatants were harvested after up to 24 h of co-culture of T cell clone T4.8-1-29 with K562-A2, Mel-624.38, Colo-678, 647-V and SuDHL-6 (all HLA-A*02^{pos}). Specific IFN-gamma secretion was assessed using standard ELISA.

Target Cells:
K562-A2 (HLA-A*02^{pos}, PRAME^{pos})
Mel-624.38 (HLA-A*02^{pos}, PRAME^{pos})
Colo-678 (HLA-A*02^{pos}, PRAME^{neg})
647-V (HLA-A*02^{pos}, PRAME^{neg})
SuDHL-6 (HLA-A*02^{pos}, PRAME^{neg})

### Results

T cell clone T4.8-1-29 showed high IFN-gamma secretion in co-culture with PRAME^{pos}, HLA-A*02^{pos} tumor cell lines K562-A2 and Mel-624.38 (positive control: peptide-pulsed T2 cells)

No PRAME^{neg}, HLA-A*02^{pos} tumor cell lines were recognized by T cell clone T4.8 (negative controls; n.d., not detected).

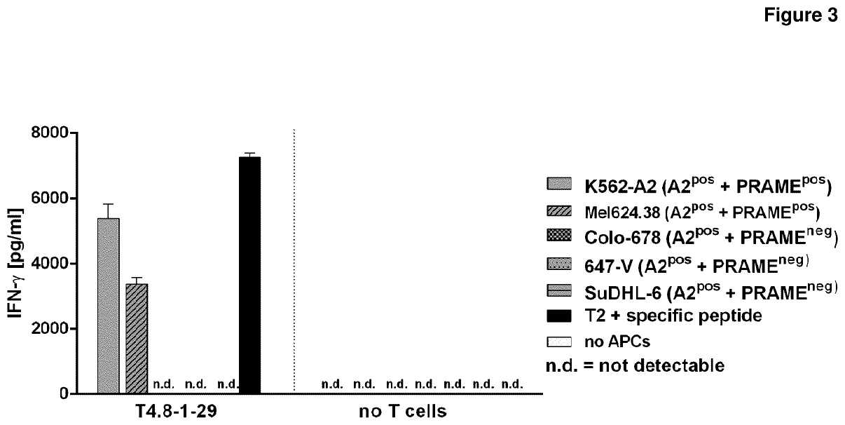

Only tumor cell lines expressing HLA-A*02 and PRAME were recognized by the self-restricted T cell clone T4.8-1-29, indicating antigen-specificity (FIG. 3).

### Example 2.3: Functional Avidity

Experimental Layout: Stimulation with Peptide-Pulsed T2 Cells

Functional T cell avidity for PRAME₁₀₀₋₁₀₈ (VLD) peptide recognition was measured by detection of IFN-gamma secretion after co-culturing clone T4.8-1-29 with peptide-loaded T2 cells.

Target cells: T2 cells (HLA-A*02^{pos}, PRAME^{neg}) loaded with titrated amounts of exogenous PRAME₁₀₀₋₁₀₈ (VLD) peptide (10⁻⁵ M to 10⁻¹² M).

Effector-to-target ratio (E:T) of 1:1.

The relative IFN-gamma release is displayed in percentage of maximum release.

The half-maximal IFN-gamma secretion defining the functional avidity is indicated by dashed lines.

Culture supernatants were harvested after ˜24 h of co-culture and assessed by standard ELISA.

### Results

Clone T4.8-1-29 showed half-maximal IFN-gamma secretion at between about 1×10⁻⁹ and 1×10⁻¹⁰ mol/L [M] concentration of PRAME₁₀₀₋₁₀₈ peptide (mean of two independent experiments), which lies within the physiological range of virus-specific T cells and is reported to represent the desired functional avidity for efficient anti-tumor efficacy (Aleksic, M et al. Eur. J. Immunol.; 42 (12):3174-3179).

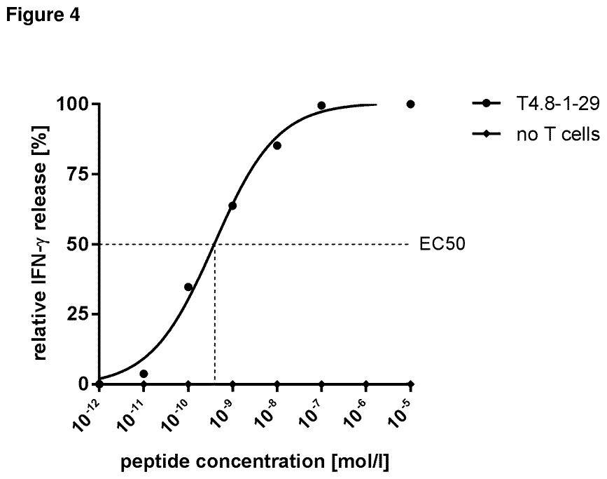

→TCR T4.8-1-29: ˜1×10⁻⁹ mol/L [M] (FIG. 4)

### Example 2.3: Analysis of Transgenic T Cell Receptor: TCR T4.8-1-29

Having identified PRAME¹⁰⁰⁻¹⁰⁸-specific T cell clone T4.8-1-29, next steps involved isolation of the DNA sequence information coding for the corresponding TCR chains, transfer of the cloned TCR into adequate recipient T cells and subsequent functional analysis of the TCR-engineered T cells.

### Example 2.3.1: T Cell Receptor Sequence Analysis

DNA sequences of the original clone T4.8-1-29 TCR alpha and beta chains were analyzed in-house by next generation sequencing (NGS-TCRseq). Corresponding TCR alpha and beta DNA sequences were reconstructed by DNA gene synthesis (GeneArt, Regensburg) and cloned into pGEM vector backbones for ivtRNA production as well as retroviral vectors for stable transduction.

### Example 2.3.2: Functional Validation of Transgenic TCR

Transfer of TCR Sequence of T Cell Clone T4.8-1-29 into Recipient Cells

TCR DNA sequences of original T cell clone T4.8-1-29 were either in vitro transcribed into RNA encoding the full T4.8-1-29 TCR sequences for transient transfection of recipient effector cells by electroporation, or used for stable transduction of effector cells by using retroviral vector constructs, also encoding the full TCR T4.8-1-29 sequence.

### Experimental Layout: Stimulation by Peptide-Pulsed T2 Cells

Specific IFN-gamma secretion of TCR T4.8-1-29-transfected recipient T cells (CD8^{pos} recipient T cell clone+T4.8-1-29 ivtRNA) in co-culture with PRAME₁₀₀₋₁₀₈ (VLD) peptide-pulsed T2 cells was measured using standard ELISA.

Target cells: T2 cells (HLA-A*02^{pos}, PRAME^{neg}) pulsed with 10⁻⁵ M VLD (relevant) or “ELA peptide” (irrelevant) peptide (ELAGIGILTV, MelanA, SEQ ID NO: 231).

### Results

TCR T4.8-1-29-transfected recipient T cells showed good recognition of T2 cells loaded with relevant peptide but no recognition when T2 cells were loaded with irrelevant peptide.

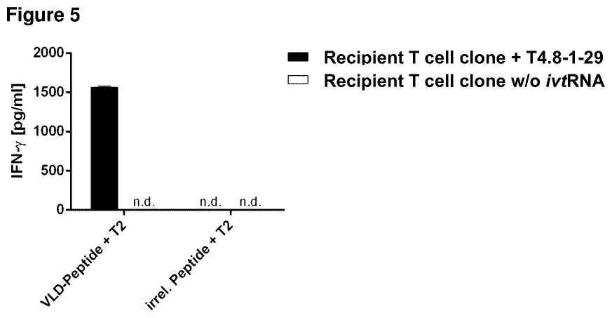

T4.8-1-29 TCR alpha and beta chain DNA sequences were reconstructed correctly and showed good function as transgenes (FIG. 5)

### Example 2.4: Analysis of Recognition of Self-Peptides

Experimental Layout: INF-Gamma Secretion of CD8+ Enriched PBMC Expressing TCR T4.8-1-29 on Co-Culture with Peptide Loaded T2 Cells

INF-gamma secretion of CD8+ enriched PBMC expressing the T cell receptor of clone T4.8-1-29.co-cultivated with T2 target cells (HLA-A*02^{pos}, PRAME^{neg}) loaded with 10⁻⁵ M PRAME₁₀₀₋₁₀₈ VLD peptide or ubiquitous self-peptides eluted from HLA-A*02 (131 self-peptides) was determined using ELISA-assay.

### Results

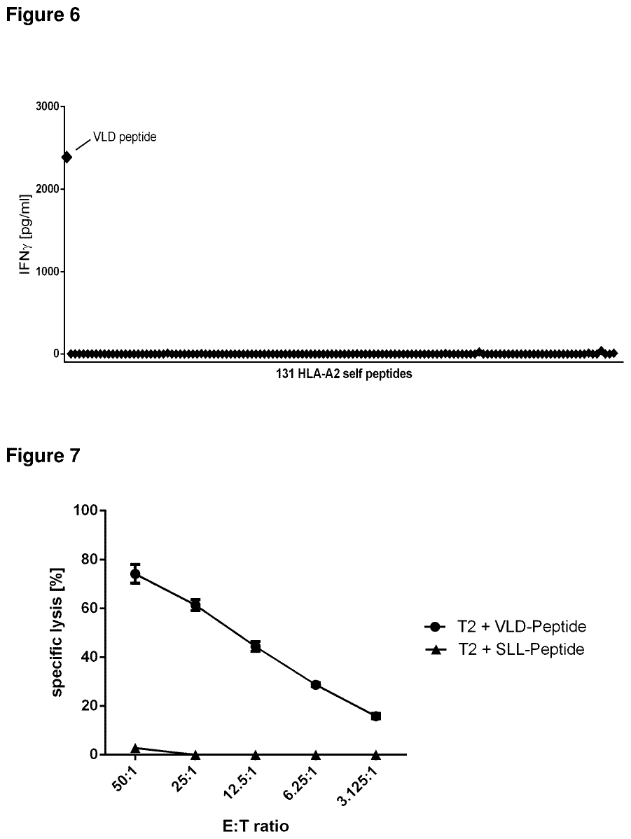

CD8⁺ enriched PBMC expressing T cell receptor of clone T4.8-1-29 show no secretion of INF-gamma if co-cultivated with T2 cells (HLA-A*02^{pos}, PRAME^{neg}) loaded with ubiquitous self-peptides (positive control: PRAME₁₀₀₋₁₀₈ loaded T2 cells) reflecting high specificity of TCR 4.8-1-29 (FIG. 6).

### Example 2.5: Cytotoxicity Analysis

### Experimental Layout: Lysis of Peptide-Pulsed T2 Cells

Lysis of PRAME₁₀₀₋₁₀₈ (VLD) peptide-pulsed T2 cells was measured by using the TVA™ fluorescent killing assay (CTL, Cellular Technology Limited, USA) determining the disappearance of fluorescently labeled target cells during co-culture with CD8 enriched PBMC expressing T4.8-1-29 TCR.

Target cells: T2 cells (HLA-A*02^{pos}, PRAME^{neg}) pulsed with 10⁻⁵ M PRAME₁₀₀₋₁₀₈ VLD (relevant) or SLL (SLLQHLIGL (SEQ ID NO: 229), PRAME, irrelevant) peptide co-cultured with TCR T4.8-1-29 expressing PBMC in graded E:T ratios

### Results

T4.8-1-29 expressing PBMC show efficient lysis of relevant (VLD) peptide-loaded T2 cells even at low E:T ratios.

T2 cells loaded with irrelevant SLL peptide (PRAME) were not lysed (negative control) at any E:T ratio (FIG. 7).

### Experimental Layout: Lysis of Tumor Cells

Cytotoxic activity against tumor cells was analyzed using the TVA™ fluorescent killing assay (CTL, Cellular Technology Limited, USA) detecting the disappearance of fluorescently labeled target cells during co-culture with PBMC expressing transgenic TCR of T cell clone T4.8-1-29.

Target cells: Human tumor cell line K562 was used for experiments. K562 cells were transfected using ivtRNA coding for human HLA-A*02:01 and/or ivtRNA coding for human PRAME. Human K562 exhibits endogenous PRAME expression (as determined by Nanostring and reported in literature). In addition, PRAME expression was increased by transfection of K562 cells with ivtRNA coding for human PRAME or by exogenous loading of PRAME₁₀₀₋₁₀₈ VLD peptide.

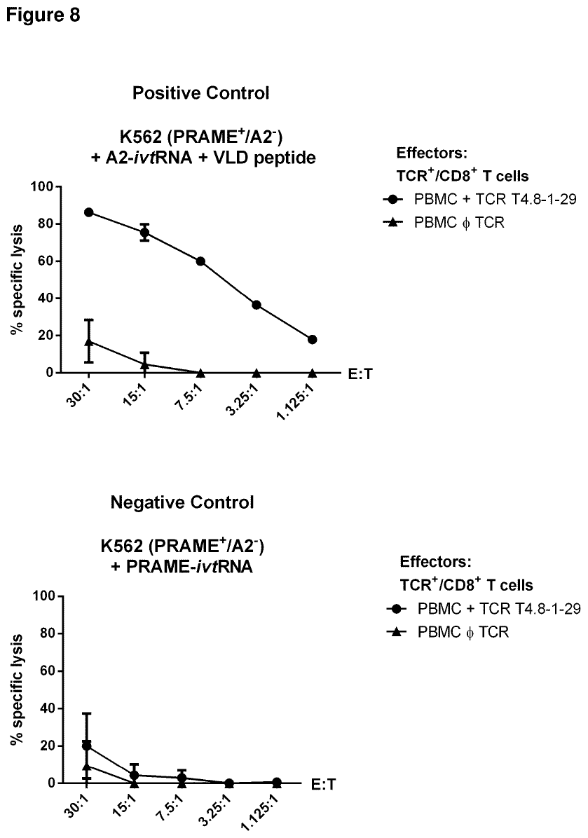

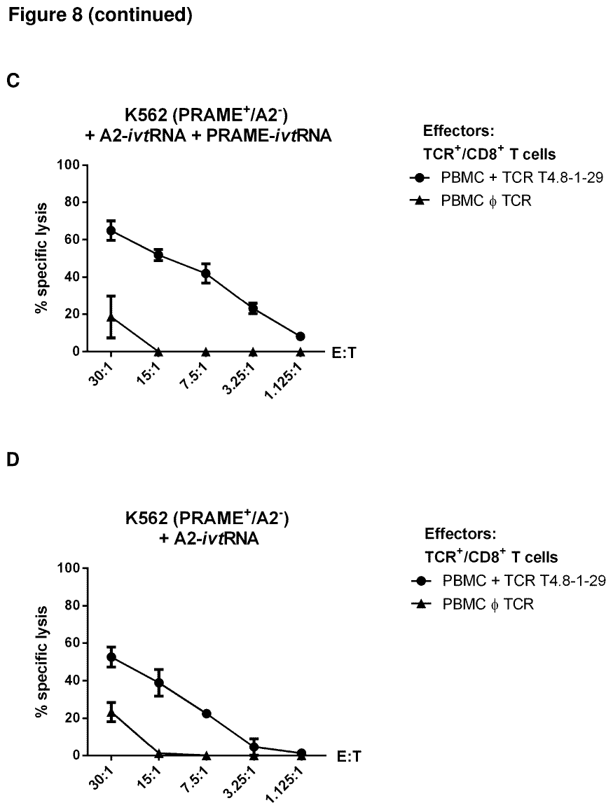

K562 transfected with ivtRNA coding for HLA-A*02:01 and loaded with PRAME₁₀₀₋₁₀₈ (VLD) peptide: K562-(PRAME⁺/A2⁻)+A2-ivtRNA+VLD peptide (FIG. 8A)

K562 transduced with ivtRNA coding for PRAME: K562-(PRAME⁺/A2⁻)+PRAME-ivtRNA (FIG. 8B)

K562 transfected with ivtRNA coding for PRAME and ivtRNA coding for HLA-A*02: K562-(PRAME⁺/A2⁻)+A2-ivtRNA (FIG. 8C)

K562 transfected with ivtRNA coding for HLA-A*02 ivtRNA: K562-(PRAME⁺/A2⁻)+A2-ivtRNA+PRAME ivtRNA (FIG. 8D)

Tumor cells were co-cultured with TCR T4.8-1-29-expressing PBMC in graded E:T ratios.

### Results

Transfection with PRAME ivtRNA as well as VLD peptide loading of HLA-A*02:01-expressing K562 cells increased specific lysis by PBMC expressing transgenic TCR T4.8-1-29 (FIG. 8A-D).

### Example 2.5: Recognition of Tumor Cells by CD8+ Enriched PBMC Expressing TCR T4.8-1-29

### Experimental Layout: Stimulation by Tumor Cell Lines

IFN-gamma ELISA was used to assess cytokine secretion after stimulation with a panel of human tumor cell lines (status of PRAME expression was detected by NanoString® nCounter® (Nanostring Technologies, Inc.) analysis).

Supernatants were harvested after up to 24 h of co-culture of CD8+ enriched PBMC expressing T cell receptor T4.8-1-29 with K562-B35, K562-A2, Mel-624.38, Colo-678 and SKMEL23. Specific IFN-gamma secretion was assessed using standard ELISA.

Target Cells:
K562-1335 (HLA-A*02^{neg}, PRAME^{pos})
K562-A2 (HLA-A*02^{pos}, PRAME^{pos})
K562-A2 (HLA-A*02^{pos}, PRAME^{pos}) loaded with VLD peptide
Mel-624.38 (HLA-A*02^{pos}, PRAME^{pos})
SkMEL23 (HLA-A*02^{pos}, PRAME^{pos})

### Results

CD8+ enriched PBMC expressing T cell receptor T4.8-1-29 showed high IFN-gamma secretion in co-culture with PRAME^{pos}, HLA-A*02^{pos} tumor cell lines K562-A2, K562-A2 additionally loaded with VLD peptide, intermediate INF-gamma secretion upon co-culture with PRAME^{pos}, HLA-A*02^{pos} Mel-624.38 and SkMEL23

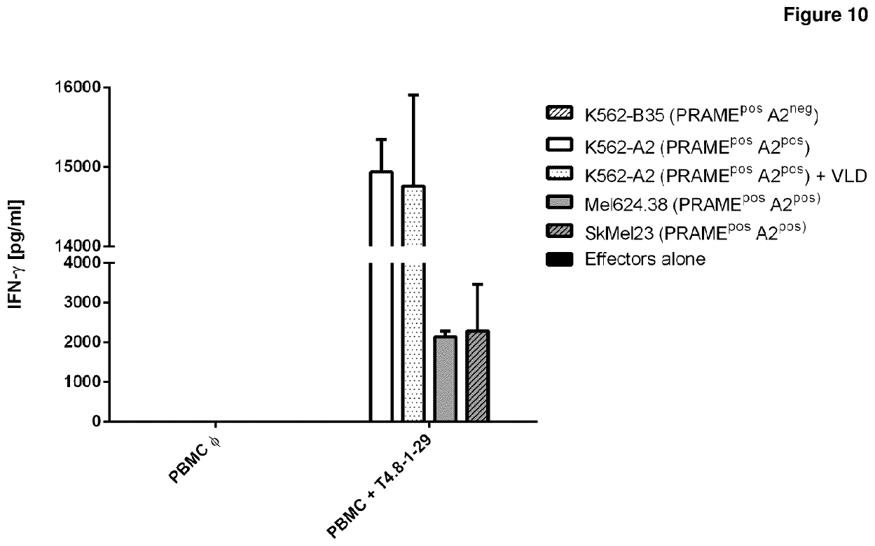

PRAME^{pos} K562 with HLA-B*35 expression did not induce INF-gamma secretion confirming HLA-A*02 restriction of TCR T4.8-1-29 (FIG. 10).

### Example 2.6: Transduction of PBMC with TCR T4.8-1-29

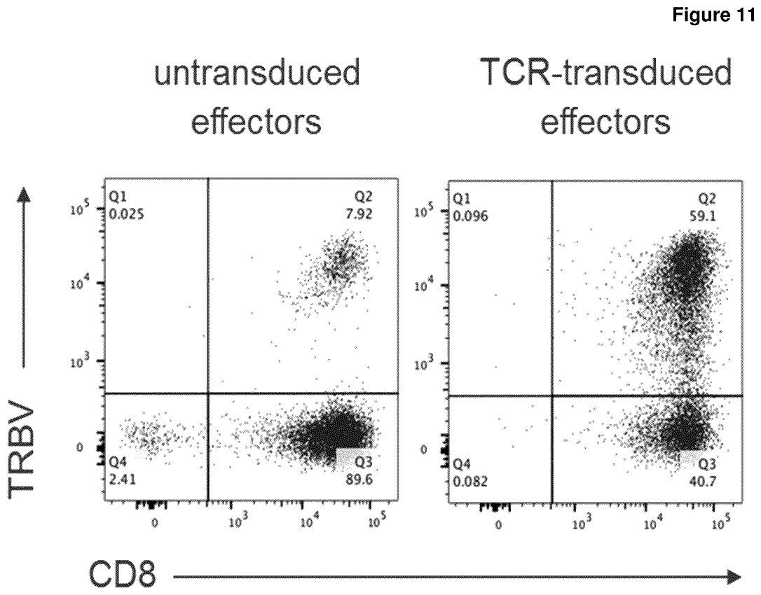

CD8 enriched PBMC of a healthy donor were transduced with a plasmid containing the TCR T4.8-1-29 construct. To analyze the TCR-transduction-efficiency, FACS analysis was performed after surface staining of untransduced and TCR T4.8-1-29-transduced PBMC. The cells were stained with antibodies specific for CD8 and the TCRs variable region of the TCR β-chain (TRBV9). In the control effector cell population, there are 8% of endogenously TRBV9-expressing T cells present, while after transduction 60% of T cells expressed TRBV9. This indicates a transduction efficiency of more than 50% (FIG. 11).

### Example 2.7: Functional T Cell Avidity for PRAME₁₀₀₋₁₀₈ (VLD) Peptide by T Cell Clone T4.8-1-29 and PBMC Transduced with TCR T4.8-1-29

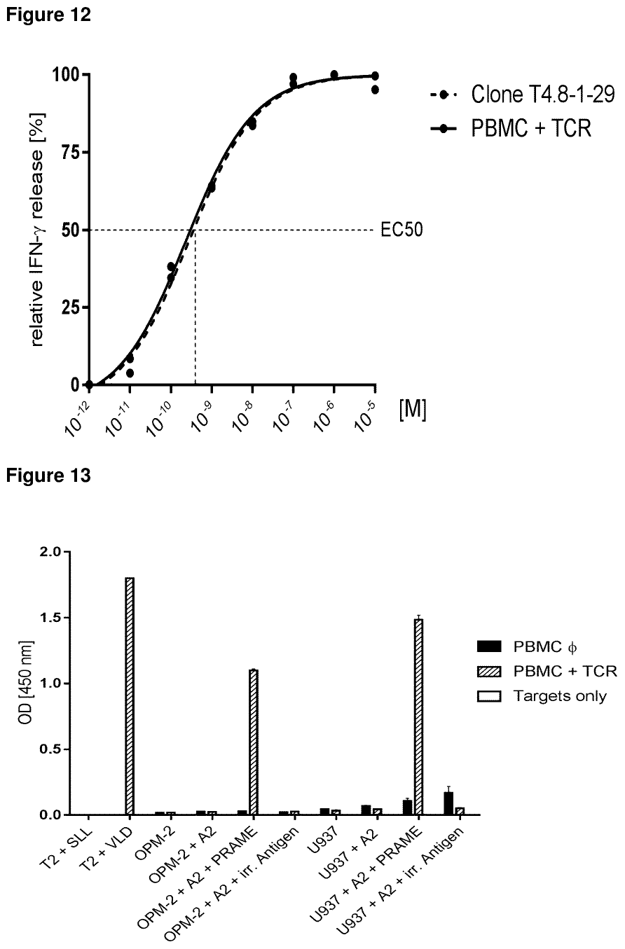

The functional T cell avidity for the PRAME₁₀₀₋₁₀₈ (VLD) peptide recognition was measured by detection of IFN-gamma secretion after co-culturing either the T cell clone T4.8-1-29 (solid curve) or effector PBMC transduced with T4.8-1-29 (dotted curve) with peptide-loaded T2 cells. The T2 cells were loaded with titrated amounts of peptide, ranging from a concentration of 10⁻⁵ M till 10⁻¹² M. The coculture-supernatants were harvested after ˜24 h of coculture and assessed by standard ELISA, the relative IFN-gamma release is displayed in percentage of maximum release. The half-maximal IFN-gamma-secretion (EC50) defining the functional avidity is indicated by the dashed line. The functional avidity of the original T cell clone and the transgenic TCR are highly similar (FIG. 12).

### Example 2.8: Analysis of Antigen Specificity of PBMC Transduced with TCR T4.8-1-29 and Untransduced Control PBMC with Different Target Cells (OPM-2 and U937)

To analyze antigen specificity, T4.8-1-29-transduced effector PBMC and untransduced control PBMC were cocultured with different target cells. The tumor cell lines OPM-2 and U937 (HLA-A2-negative and PRAME-negative) were tested either unmodified, or transfected with ivtRNA encoding HLA-A2. In addition, the cells were also tested after transfection with a combination of ivtRNA encoding for HLA-A2 and PRAME, or HLA-A2 and an irrelevant antigen. As control, the effectors were also cultured with T2 cells loaded with the PRAME_{VLD} peptide (10⁻⁵ M) or with the irrelevant PRAME_{SLL} peptide (10⁻⁵ M). After 24 h of coculture, the supernatants were harvested and secreted amounts of IFN-gamma were measured by standard ELISA. High amounts of IFN-gamma were measured for the TCR-transduced PMBC in coculture with the VLD-loaded T2 cells. Also both of the tumor cell lines transfected with HLA-A2 and the antigen PRAME induced IFN-gamma-secretion by the TCR-transduced PBMC. So only tumor cells expressing HLA-A2 as the MHC-restriction-element of need, in combination with the antigen PRAME were recognized and led to an activation of T4.8-1-29-expressing PBMC (FIG. 13),

### Example 2.9: Analysis of Antigen Specificity of PBMC Transduced with TCR T4.8-1-29 and Untransduced Control PBMC with Different Target Cells (K562, K562_A2 and Mel 624.38)

To analyze antigen specificity, T4.8-1-29-transduced effector PBMC and untransduced control PBMC were cocultured with different target cell lines. The tumor cell lines K562 (HLA-A2-negative and PRAME-positive) were tested as well as K562_A2 and Mel 624.38 (HLA-A-positive and PRAME-positive) and 647-V (HLA-A2-positive and PRAME-negative). As control, the effectors were also cultured with T2 cells loaded with the PRAME_{VLD} peptide (10⁻⁵ M) or with the irrelevant PRAME_{SLL} peptide (10⁻⁵ M). After 24 h of co-culture, the supernatants were harvested and secreted amounts of IFN-gamma were measured by standard ELISA. High amounts of IFN-gamma were measured for the TCR-transduced PMBC in coculture with the VLD-loaded T2 cells.

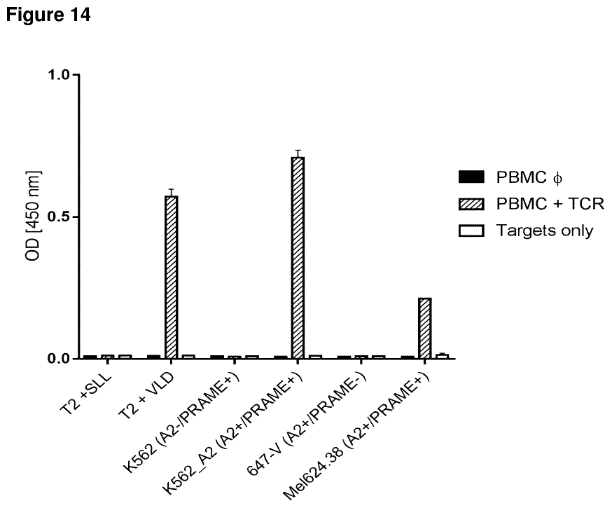

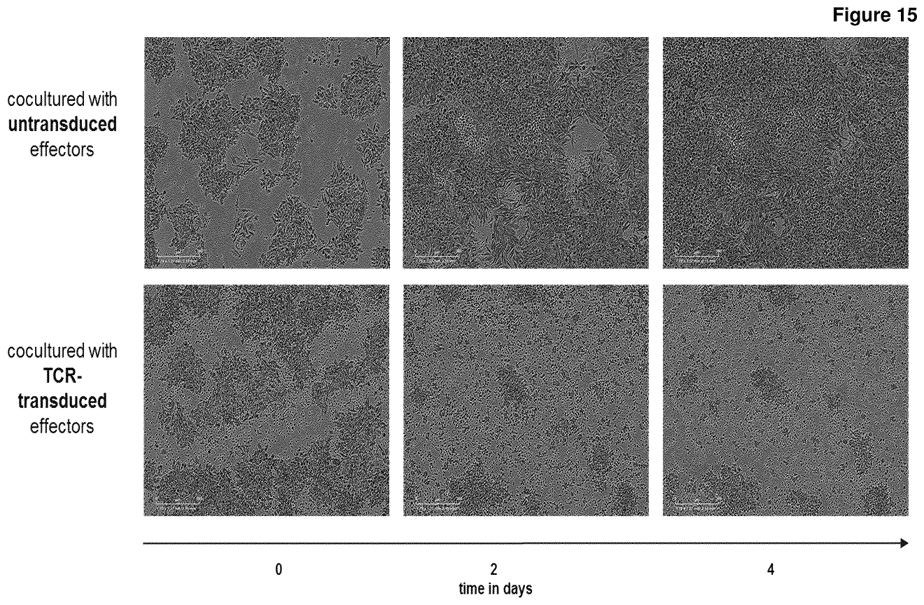

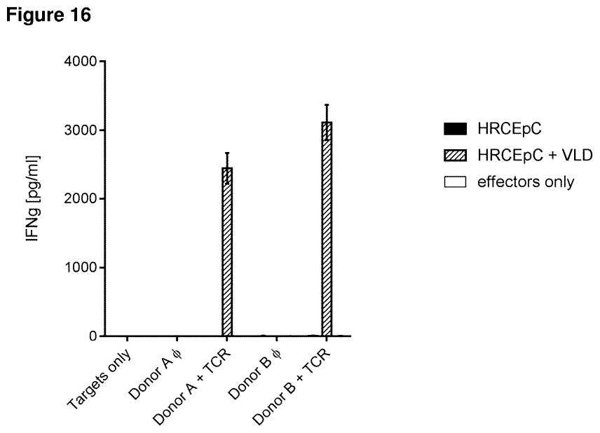

Measured IFN-gamma-values indicated activation of TCR T4.8-1-29-transduced PBMC by T2 cells loaded with the VLD-peptide and the tumor cell lines K562_A2 and Mel624.38. So only HLA-A2-positive, endogenously PRAME-expressing tumor cell lines were recognized by the transduced PBMC, while absence of either HLA-A2 or the antigen prevented activation (FIG. 14).

### Example 2.10: Analysis Cytotoxic Activity of T4.8-1-29-Transduced Effectors Against Tumor Cells

The cytotoxic activity of T4.8-1-29-transduced effectors against tumor cells was analyzed using the IncuCyte ZOOM®—Live Cell Analysis System (Essenbiosciences), a microscope-based system that allows live imaging of cells.

TCR T4.8-1-29-transduced and untransduced effector PBMC were cocultured with the HLA-A2-positive, PRAME-positive melanoma cell line Mel624.38. The melanoma cells were seeded in a 96-well plate and upon reaching a confluency of ˜60%, the effector cells were added. To visualize cell death, a red Annexin V-dye was added as well and images were taken on a daily basis for 4 days. Melanoma cell line Mel624.38 in coculture with untransduced effectors (upper row) expanded over time and only rare events of dead cells could be seen, whereas TCR-transduced effectors prevented outgrowth of tumor cells and led to the formation of cell clusters with a high amount of dying cells. This indicates, T4.8-1-29-expressing effector cells can efficiently lyse PRAME-expressing tumor cells and prevent outgrowth of tumor cells for several days.

### Example 2.11: Analysis of the Safety Profile of T4.8-1-29-Expressing PBMC

To analyze the safety profile of T4.8-1-29-expressing PBMC, the recognition of healthy human tissues has to be excluded. Therefore, T4.8-1-29-transduced PBMC derived from two different donors, were cocultured with cells derived from healthy tissues of HLA-A2-positive donors. As an example, transduced as well as untransduced PBMC were cocultured with human renal capillary epithelial cells (HRCEpC). As a control the HRCEp cells were additionally loaded with the VLD-peptide (10⁻⁵ M). After 24 h of coculture, the supernatants were harvested and secreted amounts of IFN-gamma were measured by standard ELISA. The TCR-transduced PBMC were only activated upon coculture with the peptide loaded target cells, while there was no recognition of the unmodified HRCEp cells.

1. A pharmaceutical or diagnostic composition comprising:
a nucleic acid encoding a T-cell receptor (TCR) comprising
(i) a CDR alpha chain comprising a variable region having a CDR1 consisting of the amino acid sequence VSGLRG (SEQ ID NO: 5), a CDR2 consisting of the amino acid sequence of LYSAGEE (SEQ ID NO: 3), and a CDR3 consisting of the amino acid sequence of CAVHSTAQAGTALIF (SEQ ID NO: 1);
and
(ii) a CDR beta chain comprising a variable region having a CDR1 consisting of the amino acid sequence of SGDLS (SEQ ID NO. 6), a CDR2 consisting of the amino acid sequence YYNGEE (SEQ ID NO. 4), and a CDR3 consisting of the amino acid sequence of CASSTHRGQTNYGYTF (SEQ ID NO. 2); and
a pharmaceutically acceptable excipient.

2. The pharmaceutical or diagnostic composition of claim 1, wherein the nucleic acid is a component of an expression vector, wherein the nucleic acid is operably linked to regulatory sequences configured for expression of the TCR.

3. The pharmaceutical or diagnostic composition of claim 2, wherein the expression vector is a component of a host cell, wherein the TCR expressed by the expression vector is displayed on the surface of the host cell.

4. A therapeutic or diagnostic method, comprising:
administering the pharmaceutical or diagnostic composition of claim 1 to a subject in need thereof.

5. The therapeutic or diagnostic method of claim 4, wherein the nucleic acid is administered as a component of an expression vector, wherein the nucleic acid is operably linked to regulatory sequences configured for expression of the TCR.

6. The therapeutic or diagnostic method of claim 5, wherein the expression vector is administered as a component of a host cell comprising the expression vector.
